(19) United States
(12) Patent Application Publication
(10) Pub. No.: US 2013/0274133 A1
(43) Pub. Date: Oct. 17, 2013

(54) GENETIC VARIATIONS IN THE INTERLEUKIN-6 RECEPTOR GENE AS PREDICTORS OF THE RESPONSE OF PATIENTS TO TREATMENT WITH INTERLEUKIN-6 RECEPTOR INHIBITORS

(71) Applicants: Claus Henrik Nielsen, Kgs. Lyngby (DK); Christian Enevold, Kokkkedal (DK)

(72) Inventors: Claus Henrik Nielsen, Kgs. Lyngby (DK); Christian Enevold, Kokkkedal (DK)

(73) Assignee: RIGSHOSPITALET, København Ø (DK)

(21) Appl. No.: 13/877,290

(22) PCT Filed: Sep. 30, 2011
(86) PCT No.: PCT/DK2011/050373

§ 371 Date: Jun. 24, 2013

(30) Foreign Application Priority Data
Oct. 1, 2010 (DK) PA 2010 70424
Apr. 15, 2011 (DK) PA 2011 70182

(51) Int. Cl. C12Q 1/68; A61K 39/395

(52) U.S. Cl. CPC C12Q 1/6883; A61K 39/3955
USPC 506/9; 435/6.11; 424/133.1

(57) ABSTRACT

The present invention relates to a method for predicting the response of patients to treatment with Interleukin-6 Receptor (IL6R) inhibitors, such as antibodies directed against the IL6R. The method comprises the analysis of one or more genetic variations, in particular single nucleotide polymorphisms, in or associated with the Interleukin-6 Receptor gene. The present invention further relates to a kit for use in predicting the response of patients to treatment with IL6R inhibitors, such as Tocilizumab. The patients suffers from rheumatoid arthritis.

### FIELD OF INVENTION

The present invention relates to a method for predicting the response of patients to treatment with Interleukin-6 Receptor (IL6R) inhibitors, such as antibodies directed against the IL6R. The method comprises the analysis of one or more genetic variations in or associated with the Interleukin-6 Receptor gene. The present invention further relates to a kit for use in predicting the response of patients to treatment with IL6R inhibitors, such as Tocilizumab.

### BACKGROUND OF INVENTION

Tocilizumab is a humanized recombinant IgG₁ₖ monoclonal antibody directed against the human interleukin-6-receptor (IL-6R, CD126). It is the first biologic agent specifically targeting IL-6 signalling. IL-6 is a cytokine with diverse effects on many cells relevant to a number of diseases and conditions including rheumatoid arthritis (RA). Tocilizumab is given to patients with moderate to severe rheumatoid arthritis as intravenous injections every 4 weeks at a dose of 4 mg/kg, which can be increased to 8 mg/kg depending on clinical response.

Tocilizumab was approved by the FDA on Jan. 8, 2010 for the treatment of rheumatoid arthritis. In the USA, it is currently approved only for adults with moderate to severely active RA who have not responded to one or more anti-TNF therapies. The U.S. approval was based on five pivotal trials and other experience. Tocilizumab has previously been approved for clinical use in Japan and Europe.

In the five pivotal trials, Tocilizumab demonstrated clinical efficacy in a number of RA populations; but the fraction of non-responders ranges from 30-50%, hence a large quantity of RA patients have no response to the drug.

It is estimated that 5.2 million patients with moderate to severe rheumatoid arthritis exist within the 7 major markets (USA, Japan, Italy, Germany, Spain, United Kingdom and France). Many more potential users with other autoimmune diseases exist. The cost of Tocilizumab per patient is estimated to be $1,060 to $2,125 per month, depending on the dose. Thus, Tocilizumab has a forecasted sale of 1,000,000,000 USD.

Generally, response to therapy with biological pharmaceuticals is likely to be governed by several factors, including genetic variations. However, the genetic factors involved in determining the outcome of treatment with existing biologicals have yet to be identified although several attempts have illustrated the complex nature of this issue. In rheumatoid arthritis, previous studies have suggested the involvement of several genetic polymorphisms in determining the outcome of anti-TNF therapy.

Hasan et al. and Marinou et al. have hypothesized about the possible existence of a relationship between the SNP rs2228145 (also known as rs8192284) and the clinical outcome to TCZ therapy.

Besides TCZ, there are a number of IL6R inhibitors, such as monoclonal antibodies directed against the IL6R currently undergoing clinical trials.

### SUMMARY OF INVENTION

The present invention relates to a method for predicting the response of patients to treatment with IL6R inhibitors, such as antibodies directed against the IL6R. In a preferred embodiment the antibody of the present invention is Tocilizumab (TCZ). The method comprises the analysis of one or more genetic variations in or associated with the Interleukin-6 Receptor gene.

In one embodiment, the present invention relates to a method for predicting the response of a patient to treatment with an IL6R inhibitor such as an antibody against the IL6R comprising the steps of
i) obtaining a sample of genomic DNA from the patient,
ii) detecting at least one genetic variation in the Interleukin 6 Receptor gene,
iii) predicting whether said patient is a responder, non-responder or a partial responder.

In a preferred embodiment, the IL6R inhibitor is an IL6R antibody, such as the FDA-approved monoclonal antibody Tocilizumab.

Several different classes of genetic variations exist, e.g. Single Nucleotide Polymorphisms (SNPs), copy-number variations (CNV), insertions/deletions (indels), and microsatellites. Genetic variations according to the present invention include but are not limited to, SNPs, CNVs, indels and microsatellites.

The present invention further relates to a kit for use in predicting the response of patients to treatment with IL6R inhibitors, such as Tocilizumab.

### DESCRIPTION OF DRAWINGS

FIG. 1. Display of residues of the third extracellular domain of the interleukin-6 receptor influenced strongly by binding of IL-6. A, the results of the chemical shift mapping data were translated onto the crystal structure of IL-6R-D3. Strands are labeled A-G, and residues that appear to undergo a significant structural change upon binding IL-6 are in dark grey. B, orientation of IL-6 and receptor IL-6RD2D3 relative to each other as displayed in the crystal structure. Residues of IL-6R-D3 with chemical shift differences 0.03 ppm are marked in dark grey. From: Schwantner et al., J Biol Chem 2004; 279: 571-6.

FIG. 2. Three-dimensional structural model of the cytokine receptor family domain of human IL-6R. The wide arrows represent the presumed, 3-strands, and narrow strands represent the presumed loops as predicted by Bazan (1990). The numbers indicate the positions of some amino acid residues. Small-dotted patches, amino acid residues showing no influences after substitution; large-dotted patches, amino acid residues required for binding IL-6; closed patches, amino acid residues critical for mediating the IL-6 signal but not for binding IL-6. The letters ‘C’ represent four Cys residues conserved in the cytokine receptor family, of which substitutions abolish IL-6-binding capability. The Trp-Ser-Glu-Trp-Ser motif is indicated in the one-letter code. Ig, Ig-like domain; TM, transmembrane region. From Yawata et al., EMBO Journal 1993; 12: 1705-12.

FIG. 3: The influence of the rs12083537 polymorphism on the clinical response of 43 RA patients following treatment with tocilizumab for three months. The patients were evaluated with respect to (A) the number of swollen joints, (B) the number of tender joints, (C) circulating CRP levels, and (D) DAS28-score. Statistics performed: Mann-Whitney test.

FIG. 4: Influence of the rs952146 polymorphism on the clinical response of 43 RA patients treated with tocilizumab for three months. The patients were evaluated with respect to (A) the number of swollen joints, (B) the number of tender joints, (C) circulating CRP levels, and (D) DAS28-score. Statistics performed: Kruskall-Wallis test.

FIG. 5: Influence of the rs7526293 polymorphism on the clinical response of 43 RA patients treated with tocilizumab for three months. The patients were evaluated with respect to (A) the number of swollen joints, (B) the number of tender joints, (C) circulating CRP levels, and (D) DAS28-score. Statistics performed: Kruskall-Wallis test.

FIG. 6: Influence of the two closely linked rs2228145 and rs4129267 polymorphisms on the clinical response of 43 RA patients treated with tocilizumab for three months. The patients were evaluated with respect to (A) the number of swollen joints, (B) the number of tender joints, (C) circulating CRP levels, and (D) DAS28-score. p: major allele, q: minor allele. Statistics performed: Kruskall-Wallis test.

FIG. 7: Influence of the rs4509570 polymorphism on the clinical response of 43 RA patients treated with tocilizumab for three months. The patients were evaluated with respect to (A) the number of swollen joints, (B) the number of tender joints, (C) circulating CRP levels, and (D) DAS28-score. Statistics performed: Kruskall-Wallis test.

FIG. 8: Influence of the rs10159236 polymorphism on the clinical response of 43 RA patients treated with tocilizumab for three months. The patients were evaluated with respect to (A) the number of swollen joints, (B) the number of tender joints, (C) circulating CRP levels, and (D) DAS28-score. Statistics performed: Kruskall-Wallis test.

FIG. 9: Influence of the rs4075015 polymorphism on the clinical response of 43 RA patients treated with tocilizumab for three months. The patients were evaluated with respect to (A) the number of swollen joints, (B) the number of tender joints, (C) circulating CRP levels, and (D) DAS28-score. Statistics performed: Kruskall-Wallis test.

FIG. 10. Influence of the rs7553796 polymorphisms on the clinical response of 43 RA patients treated with tocilizumab for three months. The patients were evaluated with respect to the number of swollen joints. Statistics performed: Kruskall-Wallis test.

FIG. 11: Localisation of 9 tested SNPs in the IL6R gene.

### DEFINITIONS

Allele: An allele is one of two or more forms of the DNA sequence of a particular gene. Each gene can have different alleles. Sometimes, different DNA sequences (alleles) can result in different traits, such as color. Sometimes, different DNA sequences (alleles) will have the same result in the expression of a gene. Most organisms have two sets of chromosomes, that is, they are diploid. Diploid organisms have one copy of each gene (and one allele) on each chromosome. If both alleles are the same, they are homozygote. If the alleles are different, they are heterozygotes.

Allotype: Any of the genetically determined variants of a particular antigen, which may provoke distinct immune responses.

Antigen: An antigen is a molecule recognized by the immune system.

Affinity: The term is used to denote how tightly a ligand binds to a particular target molecule.

CNVs: Copy-number variations are alterations of the DNA of a genome that results in the cell having an abnormal number of copies of one or more sections of the DNA. CNVs correspond to relatively large regions of the genome that have been deleted (fewer than the normal number) or duplicated (more than the normal number) on certain chromosomes. This variation accounts for roughly 12% of human genomic DNA and each variation may range from about one kilobase (1,000 nucleotide bases) to several megabases in size. CNVs contrast with single-nucleotide polymorphisms (SNPs), which affect only one single nucleotide base.

IL6: Interleukin-6 (IL-6) is a protein that in humans is encoded by the IL6 gene. IL-6 is an interleukin that acts as both a pro-inflammatory and anti-inflammatory cytokine. It is secreted by T cells and macrophages to stimulate immune response to trauma, especially burns or other tissue damage leading to inflammation. IL-6 is also a “myokine,” a cytokine produced from muscle, and is elevated in response to muscle contraction. It is significantly elevated with exercise, and precedes the appearance of other cytokines in the circulation. During exercise, it is thought to act in a hormone-like manner to mobilize extracellular substrates and/or augment substrate delivery. Additionally, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10. IL-6 is one of the most important mediators of fever and of the acute phase response. Interleukin 6 has been shown to interact with interleukin-6 receptor and glycoprotein 130. IL-6 signals through a cell-surface type I cytokine receptor complex consisting of the ligand-binding IL-6Rα chain (CD126), and the signal-transducing component gp130 (also called CD130).

IL6R: The Interleukin 6 receptor (IL6R) is also known as CD126 (Cluster of Differentiation 126) and is a type I cytokine receptor. The IL6 receptor is a protein complex consisting of an IL-6 receptor subunit (IL6R) and interleukin 6 signal transducer Glycoprotein 130.

Indels (insertions/deletions): Indels, as defined as either an insertion or deletion, can be used as genetic markers. Indels describe a special mutation class, defined as a mutation resulting in a colocalised insertion and deletion resulting in a colocalized insertion and deletion and a net gain or loss in nucleotides. A microindel is defined as an indel that results in a net gain or loss of 1 to 50 nucleotides. In coding regions of the genome, unless the length of an indel is a multiple of 3, they produce a frameshift mutation. Indels can be contrasted with a point mutation; where an Indel inserts or deletes nucleotides from a sequence, a point mutation is a form of substitution that replaces one of the nucleotides.

Microsatellites: Microsatellites are also known as Simple Sequence Repeats (SSRs) or sometimes Short Tandem Repeats (STRs). They are repeating sequences of 1-6 base pairs of DNA. Microsatellites can be used as molecular markers.

SNP: A single-nucleotide polymorphism (SNP, pronounced snip) is a DNA sequence variation occurring when a single nucleotide—A, T, C, or G—in the genome (or other shared sequence) differs between members of a species or paired chromosomes in an individual. For example, two sequenced DNA fragments from different individuals, AAGCCTA to AAGCTTA, contain a difference in a single nucleotide. In this case we say that there are two alleles: C and T. Almost all common SNPs have only two alleles.

Within a population, SNPs can be assigned a minor allele frequency—the lowest allele frequency at a locus that is observed in a particular population. This is simply the lesser of the two allele frequencies for single-nucleotide polymorphisms. There are variations between human populations, so a SNP allele that is common in one geographical or ethnic group may be much rarer, or even absent, in another. Single nucleotide polymorphisms may fall within coding sequences of genes, non-coding regions of genes, or in the intergenic regions between genes. SNPs that are not in protein-coding regions may still have consequences for gene splicing, transcription factor binding, or the sequence of non-coding RNA. As there are for genes, there are also bioinformatics databases for SNPs. dbSNP is a SNP database from National Center for Biotechnology Information (NCBI).

Gp130: Glycoprotein 130 (also known as gp130, IL6ST, IL6-beta or CD130) is a transmembrane protein which is the founding member of the class of all cytokine receptors. It forms one subunit of type I cytokine receptors within the IL-6 receptor family. It is often referred to as the common gp130 subunit, and is important for signal transduction following cytokine engagement. As with other type I cytokine receptors, gp130 possesses a WSXWS amino acid motif that ensures correct protein folding and ligand binding. It interacts with Janus kinases to elicit an intracellular signal following receptor interaction with its ligand. Structurally, gp130 is composed of five fibronectin type-III domains and one immunoglobulin-like C2-type (immunoglobulin-like) domain in its extracellular portion. The members of the IL-6 receptor family all complex with gp130 for signal transduction. For example, IL-6 binds to the IL-6 Receptor. The complex of these two proteins then associates with gp130. This complex of 3 proteins then homodimerizes to form a hexameric complex which can produce downstream signals.

Tocilizumab: Tocilizumab (TCZ) is a humanized monoclonal antibody against the interleukin-6 receptor (IL-6R) used as an immunosuppressive drug, mainly for the treatment of rheumatoid arthritis.

Linkage: Genetic linkage is a term which describes the tendency of certain loci or alleles to be inherited together. Genetic loci on the same chromosome are physically close to one another and tend to stay together during meiosis, and are thus genetically linked.

Linkage disequilibrium: In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). Numerically, it is the difference between observed and expected (assuming random distributions) allelic frequencies.

Drug response: A response to a drug may be complete, partial or absent. When a response is complete, the disease symptoms are either completely gone or almost completely gone. As a rule of thumb, if the disease symptoms are decreased by more than approximately 90%, the response may be considered complete. Partial response implies that the drug response is not complete. As a rule of thumb, a reduction of disease by 30% or more on clinical examination or by other means may be considered a partial response. Patients who have no (absent) response are non-responders. As a rule of thumb, non-responders have less than 30% reduction in disease symptoms. A partial responder to TCZ according to the present invention is thus a patient exhibiting approximately a 30-90% reduction in disease symptoms.

### DETAILED DESCRIPTION OF THE INVENTION

Patients with rheumatoid arthritis usually undergo approximately 3 months of treatment with Tocilizumab and other biologics, corresponding to a cost of >3000 USD, before treatment failure is recognized by the clinician. A test with the ability to distinguish responders from non-responders would thus save payers within the health care system more than 2850 USD per patient, provided that the test costs 100-150 USD.

With more than 5 million potential users (patients with moderate to severe RA) within the 7 major markets alone, and >30% non-responders to Tocilizumab, the test could literally save the health care systems in these countries billions of USD spent on ineffective therapy.

Aside from the economical considerations, the non-responding patients could avoid suffering from RA symptoms and potential worsening of the disease during the period of ineffective therapy, by the administration of an alternative treatment.

The above considerations to both patients and economy indicate that there is indeed a true need for a test capable of predicting whether a patient is likely to benefit from TCZ treatment, to avoid treating patients with TCZ, which will have no benefit from the drug.

As the above is also true for other IL6R inhibitors, such as IL6R antibodies different from TCZ, the present invention relates to a method for predicting the response of patients to treatment with Interleukin-6 Receptor (IL6R) inhibitors, such as antibodies directed against the IL6R.

The method comprises the analysis of one or more genetic variations in or associated with the Interleukin-6 Receptor gene. Preferably, the IL6R inhibitor of the present invention is an antibody such as Tocilizumab (TCZ). The patients are in one embodiment rheumatoid arthritis patients, however this patient group should not be construed as limiting for the present invention as the methods of the present invention may be used to predict the response of all patients undergoing treatment with an IL6R inhibitor, irrespective of the reasons for receiving treatment with an IL6R inhibitor, i.e. the underlying disease.

The present invention further relates to a kit for use in predicting the response of patients to treatment with IL6R inhibitors, such as Tocilizumab.

At least in some instances it is important that such a method is capable of distinguishing between non-responders and partial responders to the IL6R inhibitor, such as TCZ to avoid the exclusion of partial responders from receiving therapy. Partial responders may have great benefit of receiving treatment with an IL6R inhibitor such as TCZ, particularly in cases where no other drugs are capable of providing adequate symptom relief.

In one embodiment, the present invention relates to a method for predicting the response of a patient to treatment with an IL6R inhibitor such as an antibody against the IL6R comprising the steps of
i) obtaining a sample of genomic DNA from the patient,
ii) detecting at least one genetic variation in the Interleukin 6 Receptor gene,
iii) predicting whether said patient is a responder, non-responder or a partial responder.

In another embodiment, the present invention relates to a method of discriminating between responders and non- and/or partial responders to treatment with an IL6R inhibitor such as an antibody against the IL6R comprising the steps of
i) obtaining a sample of genomic DNA from a patient,
ii) detecting at least one genetic variation in the Interleukin 6 Receptor gene,
iii) predicting whether said patient is a responder, a non-responder or a partial responder.

In yet another embodiment, the present invention relates to a method of discriminating between non-responders and partial responders to treatment with an IL6R inhibitor such as an antibody against the IL6R comprising the steps of
i) obtaining a sample of genomic DNA from a patient,
ii) detecting at least one genetic variation the Interleukin 6 Receptor gene,
iii) predicting whether said patient is a responder, a non-responder or a partial responder.

Preferably, the IL6R inhibitor of the present invention is an antibody against the IL6R, such as Tocilizumab, however, the methods of the present invention may be used to predict the response of patients to other IL6R inhibitors, some of which are currently undergoing clinical trials.

The methods of the present invention thus relate to methods for determining whether a patient should be treated with an inhibitor of the IL6R, such as Tocilizumab or not. The methods may be used both in determining the group of patients which are likely to benefit from treatment with the IL6R inhibitor and patients which should be excluded from treatment with the IL6R inhibitor.

Genetic variations of the present invention include, but are not limited to SNPs, CNVs, indels and microsatellites. In a particular embodiment, the genetic variation is a SNP.

In preferred embodiments, the methods of the present invention relate to in vitro diagnostic methods comprising the steps of
i) isolating genomic DNA from a sample obtained from a patient,
ii) detecting at least one genetic variation in Interleukin 6 Receptor gene,
iii) predicting whether said patient is a non-responder or a partial responder.

In one embodiment, the present invention relates to the use of Tocilizumab for treatment of a disease involving the IL6R, such as rheumatoid arthritis, multiple myeloma, juvenile chronic arthritis, osteoarthritis, asthma, Crohn's disease, interstitial lung disease, inflammatory bowel disease, systemic sclerosis, intraocular inflammation, Graves' disease and endometriosis in a patient predicted to be responder or a partial responder according to the methods of the present invention.

The patients are in a preferred embodiment rheumatoid arthritis patients, however, the method of the present invention may be used to predict the response of all patients with a disease involving the IL6R, said disease being treatable or considered for treatment with an IL6R inhibitor, such as the IL6R antibody Tocilizumab.

### Tocilizumab (TCZ)

Tocilizumab (INN, or atlizumab, developed by Hoffmann-La Roche and Chugai under the trade names Actemra and RoActemra) is a humanized monoclonal antibody against the interleukin-6 receptor (IL-6R) used as an immunosuppressive drug, mainly for the treatment of rheumatoid arthritis. Interleukin 6 is a cytokine that plays an important role in the immune response and is implicated in the pathogenesis of many diseases, such as autoimmune diseases, multiple myeloma and prostate cancer.

The outcome of TCZ therapy is most likely determined by several different genetic variations, as well as non-genetic factors.

Patients may have no response, partial or impaired response or complete or full response to TCZ. A non-responder exhibits less than approximately 30% reduction in disease symptoms. A partial responder exhibits approximately 30-90% reduction in disease symptoms and a complete responder exhibits more than approximately 90% reduction in disease symptoms in response to TCZ treatment.

A number of other IL6R antibodies are currently undergoing clinical trials. The methods of the present invention also apply to such other IL6R antibodies.

### Rheumatoid Arthritis

In one embodiment, the present invention relates to a method for predicting the response of rheumatoid arthritis patients to treatment with an IL6R inhibitor, such as the IL6R antibody TCZ.

Rheumatoid arthritis (RA) is a chronic, systemic inflammatory disorder that may affect many tissues and organs, but principally attacks synovial joints. The process produces an inflammatory response of the synovium (synovitis) secondary to hyperplasia of synovial cells, excess synovial fluid, and the development of pannus in the synovium. The pathology of the disease process often leads to the destruction of articular cartilage and ankylosis of the joints. Rheumatoid arthritis can also produce diffuse inflammation in the lungs, pericardium, pleura, and sclera, and also nodular lesions, most common in subcutaneous tissue under the skin. Although the cause of rheumatoid arthritis is unknown, autoimmunity plays a pivotal role in both its chronicity and progression, and RA is considered a systemic autoimmune disease.

About 1% of the world's population is afflicted by rheumatoid arthritis, women three times more often than men. Onset is most frequent between the ages of 40 and 50, but people of any age can be affected. It can be a disabling and painful condition, which can lead to substantial loss of functioning and mobility if not adequately treated. It is a clinical diagnosis made on the basis of symptoms, physical exam, radiographs (X-rays) and lab tests; although the American College of Rheumatology (ACR) and the European League Against Rheumatism (EULAR) publish diagnostic guidelines. Diagnosis and long-term management are typically performed by a rheumatologist, an expert in auto-immune diseases.

Various treatments are available. Non-pharmacological treatment includes physical therapy, orthoses, occupational therapy and nutritional therapy but none stop progression of joint destruction. Analgesia (painkillers) and anti-inflammatory drugs, including steroids, are used to suppress the symptoms, while disease-modifying anti-rheumatic drugs (DMARDs) are required to inhibit or halt the underlying immune process and prevent long-term damage. In recent times, the newer group of biologic medicines has increased treatment options.

### The Effect of Tocilizumab on Other Diseases

In addition to its approved use in RA, Tocilizumab may find a place in the treatment of the following diseases that are all associated with perturbed or elevated soluble IL-6R levels: colitis ulcerosa, morbus Chron, juvenile chronic arthritis, multiple myeloma, osteoarthritis, asthma, interstitial lung disease, systemic sclerosis, intraocular inflammation, Graves' disease, endometriosis, multiple sclerosis, schizophrenia and mania, and psychological stress, cf. table 1.

The method of the present invention may be used to predict whether patients suffering from a disease, which may be treated with TCZ, will actually respond to and benefit from TCZ treatment.

In one embodiment, the present invention relates to a method for predicting the response of patients to TCZ, said patients suffering from a disease involving the IL6R.

In another embodiment, the present invention relates to a method for predicting the response of patients to TCZ, said patients suffering from a disease which is treatable with TCZ.

In yet another embodiment, the present invention relates to a method for predicting the response of patients having colitis ulcerosa, morbus Crohn, juvenile chronic arthritis, multiple myeloma, osteoarthritis, asthma, interstitial lung disease, systemic sclerosis, intraocular inflammation, Graves' disease, endometriosis, multiple sclerosis, schizophrenia and mania, or psychological stress to TCZ.

TABLE 1

| | | |
|---|---|---|
| Clinical condition | sIL-6R | Soluble IL-6R levels (Alpha subunit) |

Cancers

| | | |
|---|---|---|
| Multiple myeloma | ▴▾ | The concentrations of sIL-6R were significantly |
| | | higher in the patients who died within 3 years |
| | | compared with those who survived. |

Inflammatory disorders

| | | |
|---|---|---|
| Rheumatoid arthritis | ▴ | Elevated sIL-6R levels in the sera of patients |
| | | were higher than those of the control group. |
| Juvenile chronic arthritis | ▴ | Significant increases in both IL-6 and sIL-6R, |
| | | which correlated with fever |
| Osteoarthritis | ▴ | Elevated levels, but not to the extent associated |
| | | with rheumatoid arthritis |
| Asthma | ▴ | Asthmatic patients have high serum sIL-6R |
| | | levels |
| Crohn's disease | ▴ | Increased levels of IL-6 and sIL-6R have been |
| | | demonstrated in both serum and intestinal |
| | | tissues of the patients |
| Interstitial lung disease | ▴ | Raised sIL-6R levels contribute to systemic and |
| | | local responses in pneumonia and sarcoidosis |
| | | patients |
| Inflammatory bowel | ▴ | Elevated IL-6 and sIL-6R levels contribute to |
| disease | | the pathogenesis of chronic intestinal |
| | | inflammation |
| Systemic sclerosis | ▴ | sIL-6R levels correlate with the severity of |
| | | pulmonary fibrosis associated with systemic |
| | | sclerosis |
| Intraocular inflammation | ▴ | Elevated sIL-6R and IL-6 levels in the aqueous |
| | | humor of uveitis patients |
| Graves' disease | ▴ | An increased was observed in the serum |
| | | concentration of sIL-6R in the course of Graves' |
| | | disease. |
| Endometriosis | ▴ | Peritoneal and serum sIL-6R levels significantly |
| | | higher than in other benign gynecologic |
| | | conditions |

Neurological conditions

| | | |
|---|---|---|
| Multiple sclerosis | ▴ | Elevated serum sIL-6R concentrations correlate |
| | | with disease severity |
| Schizophrenia and mania | ▴ | Higher sIL-6R levels in psychotic patients than |
| | | healthy volunteers |
| Psychological stress | ▴ | Elevated in post-traumatic stress disorder, |
| | | especially in patients with major depression |

### IL6R Inhibitors for Treatment of Particular Patient Groups

Tocilizumab is currently administered to patients on a trial and error basis, meaning that the drug is given to all patients, despite the fact that a lot of the treated patients will have no or very little benefit from the drug.

The method of the present invention allows the prediction of the particular patient group, which will benefit from treatment with an IL6R inhibitor such as TCZ, said patient group being defined as the group of patients suffering from a disease involving the IL6R receptor, which will respond at least partially to treatment with the IL6R inhibitor. Thus the patient group may either exhibit a complete response or a partial response to treatment with the IL6R inhibitor.

Hence, in one embodiment, the present invention relates to Tocilizumab for treatment of a disease involving the IL6R, such as rheumatoid arthritis, multiple myeloma, juvenile chronic arthritis, osteoarthritis, asthma, Chrohn's disease, interstitial lung disease, inflammatory bowel disease, systemic sclerosis, intraocular inflammation, Graves' disease and endometriosis in a patient predicted to be responder or a partial responder to Tocilizumab treatment according to the methods of the present invention.

In a particular embodiment, the present invention relates to TCZ for use as a medicament for treatment of rheumatoid arthritis, wherein the treated patients are predicted to be at least partial responders to TCZ treatment by the methods of the present invention.

### Genetic Variations

Beside SNPs, several different classes of genetic variation exists, i.e. copy-number variations (CNV), insertions/deletions (indels), and microsatellites. These forms of genetic variations may also be accountable for the outcome of IL6R inhibitor therapy, either alone or in combination. The SNPs listed herein below account only for already known genetic variation, and as such do not necessarily cover rare or population-specific SNPs nor CNVs or indels. However, CNVs, indels, and rare SNPs may have an influence on the outcome of IL6R inhibitor therapy, e.g. one or more of the above mentioned types of genetic variation in IL6R either alone or in combination may influence the outcome of treatment with an IL6R inhibitor such as TCZ by e.g. increasing or reducing the concentration of soluble and/or membrane-bound IL-6-receptor, or by changing the conformation and/or structure of the receptor thereby affecting the ability of TCZ to neutralize it. For rare SNPs, it is conceivable that a person can have one or more rare SNPs in the IL6R region inhibiting (or improving) their response to an IL6R inhibitor such as TCZ. In this way, several persons may exhibit a poorer (or better) response to the inhibitor without necessarily carrying identical polymorphisms. Many rare or de novo polymorphisms are expected to exist but have not necessarily been reported to genetic variation databases.

Genetic variations according to the present invention include but are not limited to the below types of genetic variations:
Single nucleotide polymorphisms (SNPs)
Copy number variations (CNVs)
Insertions/deletions (indels)
microsatellites

The genetic variations are preferably localized within the IL6R gene, however, in some embodiments the genetic variations are localized close to but outside the IL6R gene.

The genetic variations according to the present invention may be localised in coding regions and non-coding regions of the IL6R gene, in the promoter region, in exons, in introns, inside the binding region of the inhibitor, outside the binding region of the inhibitor, in the upstream or downstream untranslated region.

In one embodiment, the genetic variation is localised outside the binding region of the IL6R inhibitor.

In one embodiment, the present invention relates to a method for predicting the response of a patient to treatment with an IL6R inhibitor comprising the analysis of one or more genetic variations in the IL6R gene.

In one embodiment, the present invention relates to a method for predicting responders to treatment with an IL6R inhibitor comprising the analysis of one or more genetic variations in the IL6R gene.

In one embodiment, the present invention relates to a method for predicting non-responders and/or partial responders to treatment with an IL6R inhibitor comprising the analysis of one or more genetic variations in the IL6R gene.

In one embodiment, the present invention relates to a method for predicting non-responders and/or partial responders to treatment with an IL6R inhibitor comprising the analysis of at least two genetic variations in the IL6R gene.

In one embodiment, the present invention relates to a method of discriminating between responders and non- and/or partial responders to treatment with an IL6R inhibitor.

In another embodiment, the present invention relates to a method of discriminating between non-responders and partial responders to treatment with an IL6R inhibitor.

In one embodiment, the present invention relates to a method for predicting a response to treatment with an IL6R inhibitor, such as the monoclonal antibody TCZ, comprising the analysis of one to ten genetic variations in the IL6R gene, such as two genetic variations in the IL6R gene, for example three genetic variations in the IL6R gene, such as four genetic variations in the IL6R gene, for example five genetic variations in the IL6R gene, such as six genetic variations in the IL6R gene, for example seven genetic variations in the IL6R gene, such as eight genetic variations in the IL6R gene, for example nine genetic variations in the IL6R gene, such as ten genetic variations in the IL6R gene.

### IL-6R SNPs

A number of allotypic variants exist within the IL-6-receptor. The rs2228145 SNP, formerly rs8192284 (Asp358Ala) has been a matter of investigation in other connections. Thus, the rs2228145 and “CA-repeat” polymorphisms have been associated with type 2 diabetes, obesity, metabolic syndrome, melanoma (like four other single nucleotide polymorphisms in linkage disequilibrium rs6684439, rs4845618, and rs4845622).

Approximately 40% of the Danish population carries the rs2228145 polymorphism. Only a few other single nucleotide polymorphisms have been investigated, and no investigations have been performed concerning an association between IL-6R polymorphisms and effect of Tocilizumab therapy.

The rs2228145 polymorphism has been found to influence plasma levels of the IL6R. Based on these findings, Hasan et al. and Marinou et al. have hypothesized about the possible existence of a relationship between rs2228145 and the outcome of TCZ therapy. No data relating rs2228145 to TCZ-respondership status were presented by Hasan et al. and Marinou et al. The proposed hypotheses rests solely upon previous observations that plasma levels of IL-6R are affected by the rs2228145 polymorphism.

SNP polymorphisms located in or near the IL6R may potentially change the physical appearance of the IL6R thus affecting its binding of IL6, its binding to gp130, or the affinity with which the IL6R inhibitor such as TCZ binds to the IL6R.

According to Okazaki et al., TCZ (previously known as MRA) is likely to bind within or near the pocket which binds IL6, consisting of the B′-C′ loop and the F′-G′ loop of the IL6R.

Given that different allotypes of the IL6R exist, and given that the mouse from which the MRA clone originated had been immunized with only one allotype of the IL6R, Tocilizumab would have high affinity for that particular allotype, but not necessarily for other allotypes. Even if the mouse had been immunized with a mixture of IL-6R allotypes, it might well produce different B cells that react with different IL-6R allotypes, but the monoclonal nature of Tocilizumab would still imply that a clone specific for one particular allotype has been selected.

It may therefore be hypothesized that any SNPs within, or in proximity to, the DNA sequences encoding the B′-C′ loop or the F′-G′ loop would have a high likelihood of changing the affinity of the antibody to the IL-6R. In other words, Tocilizumab may have a strong preference for binding to certain allotypes of its target molecule.

According to Yawata et al., the B′-C′ loop and the F′-G′ loop involve amino acid residues 252-55 and 297-307, hence, SNPs located in or near the regions encoding these particular amino acid residues are prime candidates for predicting the response to TCZ. In addition, SNPs which are genetically linked to such SNPs may also prove to be good candidates for predicting the response to TCZ.

The SNPs rs4129267 and rs2228145 lie within an intron in proximity to and within the exon encoding residue number 358, respectively. This is not within the extracellular region of the IL-6R, so Tocilizumab is unlikely to bind to this region of IL6R. Therefore, the association of these particular SNPs with reduced effect of Tocilizumab can probably not be explained by decreased binding of TCZ to IL6R.

Table 2 shows a number of SNPs located in or near the IL6-receptor gene. They are all considered IL6R SNPs. The SNPs listed in table 2 should not, however, be considered limiting for the present invention.

TABLE 2
SNPs located in or near the IL6-receptor gene

| Gene | SNP position | Band | Rs# | Alleles | Role | MAF |
|---|---|---|---|---|---|---|
| IL6R | chr1: 152635552 | 1q21.3 | Rs952146 | A/G | Promoter | 0.35 |
| IL6R | chr1: 152635876 | 1q21.3 | Rs17654071 | A/G | Promoter | 0.33 |
| IL6R | chr1: 152636644 | 1q21.3 | Rs6427631 | C/T | Promoter | 0.26 |
| IL6R | chr1: 152637562 | 1q21.3 | Rs2054855 | C/T | Promoter | 0.18 |
| IL6R | chr1: 152641901 | 1q21.3 | Rs4845615 | A/G | Promoter | 0.08 |
| IL6R | chr1: 152642977 | 1q21.3 | Rs1552481 | A/G | Promoter | 0.12 |
| IL6R | chr1: 152644522 | 1q21.3 | Rs4845617 | A/G | 5′ UTR | 0.42 |
| IL6R | chr1: 152646192 | 1q21.3 | Rs746475 | A/G | Intron | 0.01 |
| IL6R | chr1: 152647110 | 1q21.3 | Rs6427641 | A/G | Intron | 0.39 |
| IL6R | chr1: 152647408 | 1q21.3 | Rs11265610 | C/T | Intron | 0.18 |
| IL6R | chr1: 152647727 | 1q21.3 | Rs12083537 | A/G | Intron | 0.17 |
| IL6R | chr1: 152648673 | 1q21.3 | Rs1386821 | A/C | Intron | 0.13 |
| IL6R | chr1: 152649067 | 1q21.3 | Rs7411976 | A/C | Intron | 0.05 |
| IL6R | chr1: 152655689 | 1q21.3 | Rs4075016 | C/T | Intron | 0.01 |
| IL6R | chr1: 152655820 | 1q21.3 | Rs4075015 | A/T | Intron | 0.25 |
| IL6R | chr1: 152656365 | 1q21.3 | Rs12090237 | A/G | Intron | 0.09 |
| IL6R | chr1: 152661041 | 1q21.3 | Rs4601580 | A/T | Intron | 0.44 |
| IL6R | chr1: 152662463 | 1q21.3 | Rs6684439 | C/T | Intron | 0.32 |
| IL6R | chr1: 152666639 | 1q21.3 | Rs4845618 | G/T | Intron | 0.45 |
| IL6R | chr1: 152667423 | 1q21.3 | Rs6427658 | C/T | Intron | 0.45 |
| IL6R | chr1: 152668303 | 1q21.3 | Rs8192282 | A/G | Ala31Ala | 0.12 |
| IL6R | chr1: 152668452 | 1q21.3 | Rs11557725 | G/T | Leu81Arg | N.A. |
| IL6R | chr1: 152668596 | 1q21.3 | Rs6694817 | C/T | Intron | 0.37 |
| | | | | | (boundary) | |
| IL6R | chr1: 152669654 | 1q21.3 | Rs11557724 | C/T | Pro136Ser | N.A. |
| IL6R | chr1: 152669862 | 1q21.3 | Rs28730733 | G/T | Intron | 0.09 |
| IL6R | chr1: 152670960 | 1q21.3 | Rs7549250 | C/T | Intron | 0.45 |
| IL6R | chr1: 152671030 | 1q21.3 | Rs7553796 | A/C | Intron | 0.45 |
| IL6R | chr1: 152674043 | 1q21.3 | Rs7518199 | A/C | Intron | 0.30 |
| | | | | | (boundary) | |
| IL6R | chr1: 152674964 | 1q21.3 | Rs4845371 | C/T | Intron | 0.37 |
| IL6R | chr1: 152675724 | 1q21.3 | Rs6667434 | A/G | Intron | 0.43 |
| IL6R | chr1: 152677579 | 1q21.3 | Rs4553185 | C/T | Intron | 0.40 |
| IL6R | chr1: 152678043 | 1q21.3 | Rs4845622 | A/C | Intron | 0.26 |
| IL6R | chr1: 152680661 | 1q21.3 | Rs4393147 | C/T | Intron | 0.24 |
| IL6R | chr1: 152680710 | 1q21.3 | Rs4453032 | A/G | Intron | 0.24 |
| IL6R | chr1: 152680920 | 1q21.3 | Rs6664201 | C/T | Intron | 0.26 |
| IL6R | chr1: 152682401 | 1q21.3 | Rs4845623 | A/G | Intron | 0.37 |
| IL6R | chr1: 152685503 | 1q21.3 | Rs4537545 | C/T | Intron | 0.36 |
| IL6R | chr1: 152687402 | 1q21.3 | Rs7529229 | C/T | Intron | 0.35 |
| IL6R | chr1: 152688691 | 1q21.3 | Rs4845625 | C/T | Intron | 0.36 |
| IL6R | chr1: 152690109 | 1q21.3 | Rs4845626 | G/T | Intron | 0.27 |
| IL6R | chr1: 152692721 | 1q21.3 | Rs6689393 | A/G | Intron | 0.36 |
| IL6R | chr1: 152692888 | 1q21.3 | Rs4129267 | C/T | Intron | 0.26 |
| IL6R | chr1: 152693571 | 1q21.3 | Rs4845374 | A/T | Intron | 0.19 |
| IL6R | chr1: 152693594 | 1q21.3 | Rs2228145 | A/C | Asp358Ala | 0.35 |
| IL6R | chr1: 152693656 | 1q21.3 | Rs28730735 | C/T | Leu379Phe | N.A. |
| IL6R | chr1: 152693674 | 1q21.3 | Rs28730736 | A/G | Val385Ile | 0.10 |
| IL6R | chr1: 152696716 | 1q21.3 | Rs11265618 | C/T | Intron | 0.27 |
| IL6R | chr1: 152697442 | 1q21.3 | Rs12125954 | A/G | Intron | N.A. |
| IL6R | chr1: 152698029 | 1q21.3 | Rs10159236 | A/C | Intron | 0.17 |
| IL6R | chr1: 152698666 | 1q21.3 | Rs10752641 | C/G | Intron | 0.25 |
| IL6R | chr1: 152699044 | 1q21.3 | Rs4329505 | C/T | Intron | 0.27 |
| IL6R | chr1: 152702819 | 1q21.3 | Rs4240872 | C/T | Intron | 0.30 |
| IL6R | chr1: 152703008 | 1q21.3 | Rs4509570 | C/G | Intron | 0.25 |
| IL6R | chr1: 152703028 | 1q21.3 | Rs4341355 | C/G | Intron | 0.27 |
| IL6R | chr1: 152704520 | 1q21.3 | Rs2229238 | C/T | 3′ UTR | 0.21 |
| IL6R | chr1: 152704708 | 1q21.3 | Rs7514452 | C/T | 3′ UTR | 0.21 |
| IL6R | chr1: 152705504 | 1q21.3 | Rs4072391 | C/T | 3′ UTR | 0.26 |
| IL6R | chr1: 152706489 | 1q21.3 | Rs4379670 | A/T | 3′ UTR | 0.17 |
| IL6R | chr1: 152709584 | 1q21.3 | Rs11265621 | A/G | Downstream | 0.30 |
| IL6R | chr1: 152710833 | 1q21.3 | Rs7526293 | C/T | Downstream | 0.31 |
| IL6R | chr1: 154439338 | 1q21.3 | Rs41310931 | A/G | 3′UTR | N.A. |

Legend:
Gene, Official name for the gene containing the SNP in question;
SNP position, Chromosomal location of SNP;
Band, Chromosomal band location;
rs#, Identification number in NCBI's dbSNP database;
Alleles, Known SNP alleles;
Role, Functional role of SNP;
MAF, Expected minor allele frequency.

In one embodiment, the present invention relates to a method for predicting the response of a patient to treatment with an IL6R inhibitor comprising the analysis of one or more IL6R SNP polymorphisms.

In one embodiment, the present invention relates to a method for predicting responders to treatment with an IL6R inhibitor comprising the analysis of one or more IL6R SNP polymorphisms.

In one embodiment, the present invention relates to a method for predicting non-responders and/or partial responders to treatment with an IL6R inhibitor comprising the analysis of one or more IL6R SNP polymorphisms.

In one embodiment, the present invention relates to a method for predicting non-responders and/or partial responders to treatment with an IL6R inhibitor comprising the analysis of at least two IL6R SNP polymorphisms.

In one embodiment, the present invention relates to a method of discriminating between responders and non- and/or partial responders to treatment with an IL6R inhibitor.

In another embodiment, the present invention relates to a method of discriminating between non-responders and partial responders to treatment with an IL6R inhibitor.

In one embodiment, the present invention relates to a method for predicting the response to treatment with an IL6R inhibitor comprising the analysis of one or more IL6R SNP polymorphisms, wherein said IL6R SNP polymorphisms affect the binding affinity with which the IL6R inhibitor binds to the IL-6R.

In one embodiment, the present invention relates to a method for predicting the response to treatment with an IL6R inhibitor comprising the analysis of one or more IL6R SNP polymorphisms, wherein said IL6R SNP polymorphisms affect the binding affinity of IL6 to IL6R.

In one embodiment, the present invention relates to a method for predicting the response to treatment with an IL6R inhibitor comprising the analysis of one or more IL6R SNP polymorphisms, wherein said IL6R SNP polymorphisms affect the interaction of the IL6R with one or more interacting molecules, such as the co-receptor gp130.

In one embodiment, the present invention relates to a method for predicting the response to Tocilizumab treatment comprising the analysis of one or more IL6R SNP polymorphisms, said IL6R SNP polymorphisms being present in the coding region of the TCZ-binding domain of the IL6R, said TCZ-binding domain of the IL6R comprising amino acid residues 252-55 and 297-307 of the IL6R.

In one embodiment, the present invention relates to a method for predicting the response to treatment with an IL6R inhibitor comprising the analysis of one or more IL6R SNP polymorphisms selected from the group consisting of rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4129267, rs4845374, rs2228145, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In one embodiment, the present invention relates to a method for predicting a response to treatment with an IL6R inhibitor comprising the analysis of one to ten IL6R SNP polymorphisms, such as two IL6R SNP polymorphisms, for example three IL6R SNP polymorphisms, such as four IL6R SNP polymorphisms, for example five IL6R SNP polymorphisms, such as six IL6R SNP polymorphisms, for example seven IL6R SNP polymorphisms, such as eight IL6R SNP polymorphisms, for example nine IL6R SNP polymorphisms, such as ten IL6R SNP polymorphisms.

In one embodiment, the present invention relates to a method for predicting a response to treatment with an IL6R inhibitor comprising the analysis of two to four IL6R SNP polymorphisms, more preferred two to three IL6R SNP polymorphisms.

In one embodiment, the present invention relates to a method for predicting a response to treatment with an IL6R inhibitor comprising analysis of the IL6R SNP polymorphism rs2228145 and/or rs4129267 in combination with one or more of rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In one embodiment, the present invention relates to a method for predicting a response to Tocilizumab treatment comprising analysis of the following IL6R SNP polymorphisms and predicting the outcome of the treatment based on the following table.

| Gene | dbSNP ID | Genotype | Effect |
|---|---|---|---|
| IL6R | rs7526293 | C/T | Predicts responders |
| IL6R | rs12083537 | A/G | Predicts responders |
| IL6R | rs4509570 | C/G | Predicts responders |
| IL6R | rs2228145 | A/C | Predicts responders |
| IL6R | rs4129267 | C/T | Predicts responders |

In a particular embodiment, the present invention relates to a method for predicting a response to Tocilizumab treatment comprising analysis of the IL6R SNP polymorphism rs228145 and/or rs4129267 in combination with at least two other SNPs selected from the group consisting of rs952146, rs4075015 and rs12083537.

In a particular embodiment, the present invention relates to a method for predicting a response to Tocilizumab treatment comprising analysis of the IL6R SNP polymorphisms IL6R-rs952146, IL6R-rs12083537, and IL6R-rs4075015 either individually or in combination.

In a particular embodiment, the present invention relates to a method for predicting a response to Tocilizumab treatment comprising analysis of the IL6R SNP polymorphism

IL6R-rs2228145 in combination with IL6R-rs952146, IL6R-rs12083537, and IL6R-rs4075015.

In a particular embodiment, the present invention relates to a method for predicting a response to Tocilizumab treatment comprising analysis of the IL6R SNP polymorphism IL6R-rs4129267 in combination with IL6R-rs952146, IL6R-rs12083537, and IL6R-rs4075015.

In one embodiment the present invention relates to a method for predicting the response to Tocilizumab treatment comprising analysis of the IL6R SNP rs8192282 alone or in combination with one or more other IL6R genetic variations.

In one embodiment the present invention relates to a method for predicting the response to Tocilizumab treatment comprising analysis of the IL6R SNP rs11557725 alone or in combination with one or more other IL6R genetic variations.

In one embodiment the present invention relates to a method for predicting the response to Tocilizumab treatment comprising analysis of the IL6R SNP rs11557724 alone or in combination with one or more other IL6R genetic variations.

In one embodiment, the method of the present invention does not consist of analysing either one of the IL6R SNPs rs10159236, rs4075015 or rs7553796.

rs12083537

In one embodiment, the present invention relates to a method for predicting a response to Tocilizumab comprising analysis of the IL6R SNP polymorphism rs12083537.

In a particular embodiment, the method of the present invention consists of analyzing the rs12083537 SNP to predict the response to Tocilizumab, more specifically to predict responders to Tocilizumab.

In one embodiment, the present invention relates to a method for predicting a response to Tocilizumab treatment comprising analysis of the IL6R SNP polymorphism rs12083537 alone or in combination with one or more of rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In a particular embodiment, the present invention relates to a method for predicting a response to Tocilizumab comprising analysis of the IL6R SNP polymorphism rs12083537 in combination with rs4129267.

In a particular embodiment, the present invention relates to a method for predicting a response to Tocilizumab comprising analysis of the IL6R SNP polymorphism rs12083537 in combination with rs4075015.

In a particular embodiment, the present invention relates to a method for predicting a response to Tocilizumab comprising analysis of the IL6R SNP polymorphism rs12083537 in combination with rs952146.

In a particular embodiment, the present invention relates to a method for predicting a response to Tocilizumab comprising analysis of the IL6R SNP polymorphism rs12083537 in combination with rs17654071.

In a particular embodiment, the present invention relates to a method for predicting a response to Tocilizumab comprising analysis of the IL6R SNP polymorphism rs12083537 in combination with rs6427631.

In a particular embodiment, the present invention relates to a method for predicting a response to Tocilizumab comprising analysis of the IL6R SNP polymorphism rs12083537 in combination with rs2054855.

In a particular embodiment, the present invention relates to a method for predicting a response to Tocilizumab comprising analysis of the IL6R SNP polymorphism rs12083537 in combination with rs4845615.

In a particular embodiment, the present invention relates to a method for predicting a response to Tocilizumab comprising analysis of the IL6R SNP polymorphism rs12083537 in combination with rs1552481.

In a particular embodiment, the present invention relates to a method for predicting a response to Tocilizumab comprising analysis of the IL6R SNP polymorphism rs12083537 in combination with rs4845617.

In a particular embodiment, the present invention relates to a method for predicting a response to Tocilizumab comprising analysis of the IL6R SNP polymorphism rs12083537 in combination with rs746475.

In a particular embodiment, the present invention relates to a method for predicting a response to Tocilizumab comprising analysis of the IL6R SNP polymorphism rs12083537 in combination with rs6427641.

In a particular embodiment, the present invention relates to a method for predicting a response to Tocilizumab comprising analysis of the IL6R SNP polymorphism rs12083537 in combination with rs11265610.

In a particular embodiment, the present invention relates to a method for predicting a response to Tocilizumab comprising analysis of the IL6R SNP polymorphism rs12083537 in combination with rs2228145.

In a particular embodiment, the present invention relates to a method for predicting a response to Tocilizumab comprising analysis of the IL6R SNP polymorphism rs12083537 in combination with rs1386821.

In a particular embodiment, the present invention relates to a method for predicting a response to Tocilizumab comprising analysis of the IL6R SNP polymorphism rs12083537 in combination with rs7411976.

In a particular embodiment, the present invention relates to a method for predicting a response to Tocilizumab comprising analysis of the IL6R SNP polymorphism rs12083537 in combination with rs4075016.

In a particular embodiment, the present invention relates to a method for predicting a response to Tocilizumab comprising analysis of the IL6R SNP polymorphism rs12083537 in combination with rs12090237.

In a particular embodiment, the present invention relates to a method for predicting a response to Tocilizumab comprising analysis of the IL6R SNP polymorphism rs12083537 in combination with rs4601580.

In a particular embodiment, the present invention relates to a method for predicting a response to Tocilizumab comprising analysis of the IL6R SNP polymorphism rs12083537 in combination with rs6684439.

In a particular embodiment, the present invention relates to a method for predicting a response to Tocilizumab comprising analysis of the IL6R SNP polymorphism rs12083537 in combination with rs4845618.

In a particular embodiment, the present invention relates to a method for predicting a response to Tocilizumab comprising analysis of the IL6R SNP polymorphism rs12083537 in combination with rs6427658.

In a particular embodiment, the present invention relates to a method for predicting a response to Tocilizumab comprising analysis of the IL6R SNP polymorphism rs12083537 in combination with rs8192282.

In a particular embodiment, the present invention relates to a method for predicting a response to Tocilizumab comprising analysis of the IL6R SNP polymorphism rs12083537 in combination with rs11557725.

In a particular embodiment, the present invention relates to a method for predicting a response to Tocilizumab comprising analysis of the IL6R SNP polymorphism rs12083537 in combination with rs6694817.

In a particular embodiment, the present invention relates to a method for predicting a response to Tocilizumab comprising analysis of the IL6R SNP polymorphism rs12083537 in combination with rs11557724.

In a particular embodiment, the present invention relates to a method for predicting a response to Tocilizumab comprising analysis of the IL6R SNP polymorphism rs12083537 in combination with rs28730733.

In a particular embodiment, the present invention relates to a method for predicting a response to Tocilizumab comprising analysis of the IL6R SNP polymorphism rs12083537 in combination with rs7549250.

In a particular embodiment, the present invention relates to a method for predicting a response to Tocilizumab comprising analysis of the IL6R SNP polymorphism rs12083537 in combination with rs7553796.

In a particular embodiment, the present invention relates to a method for predicting a response to Tocilizumab comprising analysis of the IL6R SNP polymorphism rs12083537 in combination with rs7518199.

In a particular embodiment, the present invention relates to a method for predicting a response to Tocilizumab comprising analysis of the IL6R SNP polymorphism rs12083537 in combination with rs4845371.

In a particular embodiment, the present invention relates to a method for predicting a response to Tocilizumab comprising analysis of the IL6R SNP polymorphism rs12083537 in combination with rs6667434.

In a particular embodiment, the present invention relates to a method for predicting a response to Tocilizumab comprising analysis of the IL6R SNP polymorphism rs12083537 in combination with rs4553185.

In a particular embodiment, the present invention relates to a method for predicting a response to Tocilizumab comprising analysis of the IL6R SNP polymorphism rs12083537 in combination with rs4845622.

In a particular embodiment, the present invention relates to a method for predicting a response to Tocilizumab comprising analysis of the IL6R SNP polymorphism rs12083537 in combination with rs4393147.

In a particular embodiment, the present invention relates to a method for predicting a response to Tocilizumab comprising analysis of the IL6R SNP polymorphism rs12083537 in combination with rs4453032.

In a particular embodiment, the present invention relates to a method for predicting a response to Tocilizumab comprising analysis of the IL6R SNP polymorphism rs12083537 in combination with rs6664201.

In a particular embodiment, the present invention relates to a method for predicting a response to Tocilizumab comprising analysis of the IL6R SNP polymorphism rs12083537 in combination with rs4845623.

In a particular embodiment, the present invention relates to a method for predicting a response to Tocilizumab comprising analysis of the IL6R SNP polymorphism rs12083537 in combination with rs4537545.

In a particular embodiment, the present invention relates to a method for predicting a response to Tocilizumab comprising analysis of the IL6R SNP polymorphism rs12083537 in combination with rs7529229.

In a particular embodiment, the present invention relates to a method for predicting a response to Tocilizumab comprising analysis of the IL6R SNP polymorphism rs12083537 in combination with rs4845625.

In a particular embodiment, the present invention relates to a method for predicting a response to Tocilizumab comprising analysis of the IL6R SNP polymorphism rs12083537 in combination with rs4845626.

In a particular embodiment, the present invention relates to a method for predicting a response to Tocilizumab comprising analysis of the IL6R SNP polymorphism rs12083537 in combination with rs6689393.

In a particular embodiment, the present invention relates to a method for predicting a response to Tocilizumab comprising analysis of the IL6R SNP polymorphism rs12083537 in combination with rs4845374.

In a particular embodiment, the present invention relates to a method for predicting a response to Tocilizumab comprising analysis of the IL6R SNP polymorphism rs12083537 in combination with rs28730735.

In a particular embodiment, the present invention relates to a method for predicting a response to Tocilizumab comprising analysis of the IL6R SNP polymorphism rs12083537 in combination with rs28730736.

In a particular embodiment, the present invention relates to a method for predicting a response to Tocilizumab comprising analysis of the IL6R SNP polymorphism rs12083537 in combination with rs11265618.

In a particular embodiment, the present invention relates to a method for predicting a response to Tocilizumab comprising analysis of the IL6R SNP polymorphism rs12083537 in combination with rs12125954.

In a particular embodiment, the present invention relates to a method for predicting a response to Tocilizumab comprising analysis of the IL6R SNP polymorphism rs12083537 in combination with rs10159236.

In a particular embodiment, the present invention relates to a method for predicting a response to Tocilizumab comprising analysis of the IL6R SNP polymorphism rs12083537 in combination with rs10752641.

In a particular embodiment, the present invention relates to a method for predicting a response to Tocilizumab comprising analysis of the IL6R SNP polymorphism rs12083537 in combination with rs4329505.

In a particular embodiment, the present invention relates to a method for predicting a response to Tocilizumab comprising analysis of the IL6R SNP polymorphism rs12083537 in combination with rs4240872.

In a particular embodiment, the present invention relates to a method for predicting a response to Tocilizumab comprising analysis of the IL6R SNP polymorphism rs12083537 in combination with rs4509570.

In a particular embodiment, the present invention relates to a method for predicting a response to Tocilizumab comprising analysis of the IL6R SNP polymorphism rs12083537 in combination with rs4341355.

In a particular embodiment, the present invention relates to a method for predicting a response to Tocilizumab comprising analysis of the IL6R SNP polymorphism rs12083537 in combination with rs2229238.

In a particular embodiment, the present invention relates to a method for predicting a response to Tocilizumab comprising analysis of the IL6R SNP polymorphism rs12083537 in combination with rs7514452.

In a particular embodiment, the present invention relates to a method for predicting a response to Tocilizumab comprising analysis of the IL6R SNP polymorphism rs12083537 in combination with rs4072391.

In a particular embodiment, the present invention relates to a method for predicting a response to Tocilizumab comprising analysis of the IL6R SNP polymorphism rs12083537 in combination with rs4379670.

In a particular embodiment, the present invention relates to a method for predicting a response to Tocilizumab comprising analysis of the IL6R SNP polymorphism rs12083537 in combination with rs11265621.

In a particular embodiment, the present invention relates to a method for predicting a response to Tocilizumab comprising analysis of the IL6R SNP polymorphism rs12083537 in combination with rs7526293.

In a particular embodiment, the present invention relates to a method for predicting a response to Tocilizumab comprising analysis of the IL6R SNP polymorphism rs12083537 in combination with rs41310931.

rs2228145/rs4129267

In one embodiment, the present invention relates to a method for predicting a response to Tocilizumab comprising analysis of the IL6R SNP polymorphism rs2228145 or rs4129267 optionally in combination with at least one other genetic variation in the IL6R gene, such as another IL6R SNP.

In a particular embodiment, the method of the present invention does not consist of analyzing the rs2228145 SNP.

In a particular embodiment, the present invention relates to a method for predicting a response to Tocilizumab comprising analysis of the IL6R SNP polymorphism rs2228145 or rs4129267 in combination with rs4075015.

In a particular embodiment, the present invention relates to a method for predicting a response to Tocilizumab comprising analysis of the IL6R SNP polymorphism rs2228145 or rs4129267 in combination with rs952146.

In a particular embodiment, the present invention relates to a method for predicting a response to Tocilizumab comprising analysis of the IL6R SNP polymorphism rs2228145 or rs4129267 in combination with rs17654071.

In a particular embodiment, the present invention relates to a method for predicting a response to Tocilizumab comprising analysis of the IL6R SNP polymorphism rs2228145 or rs4129267 in combination with rs6427631.

In a particular embodiment, the present invention relates to a method for predicting a response to Tocilizumab comprising analysis of the IL6R SNP polymorphism rs2228145 or rs4129267 in combination with rs2054855.

In a particular embodiment, the present invention relates to a method for predicting a response to Tocilizumab comprising analysis of the IL6R SNP polymorphism rs2228145 or rs4129267 in combination with rs4845615.

In a particular embodiment, the present invention relates to a method for predicting a response to Tocilizumab comprising analysis of the IL6R SNP polymorphism rs2228145 or rs4129267 in combination with rs1552481.

In a particular embodiment, the present invention relates to a method for predicting a response to Tocilizumab comprising analysis of the IL6R SNP polymorphism rs2228145 or rs4129267 in combination with rs4845617.

In a particular embodiment, the present invention relates to a method for predicting a response to Tocilizumab comprising analysis of the IL6R SNP polymorphism rs2228145 or rs4129267 in combination with rs746475.

In a particular embodiment, the present invention relates to a method for predicting a response to Tocilizumab comprising analysis of the IL6R SNP polymorphism rs2228145 or rs4129267 in combination with rs6427641.

In a particular embodiment, the present invention relates to a method for predicting a response to Tocilizumab comprising analysis of the IL6R SNP polymorphism rs2228145 or rs4129267 in combination with rs11265610.

In a particular embodiment, the present invention relates to a method for predicting a response to Tocilizumab comprising analysis of the IL6R SNP polymorphism rs2228145 or rs4129267 in combination with rs12083537.

In a particular embodiment, the present invention relates to a method for predicting a response to Tocilizumab comprising analysis of the IL6R SNP polymorphism rs2228145 or rs4129267 in combination with rs1386821.

In a particular embodiment, the present invention relates to a method for predicting a response to Tocilizumab comprising analysis of the IL6R SNP polymorphism rs2228145 or rs4129267 in combination with rs7411976.

In a particular embodiment, the present invention relates to a method for predicting a response to Tocilizumab comprising analysis of the IL6R SNP polymorphism rs2228145 or rs4129267 in combination with rs4075016.

In a particular embodiment, the present invention relates to a method for predicting a response to Tocilizumab comprising analysis of the IL6R SNP polymorphism rs2228145 or rs4129267 in combination with rs12090237.

In a particular embodiment, the present invention relates to a method for predicting a response to Tocilizumab comprising analysis of the IL6R SNP polymorphism rs2228145 or rs4129267 in combination with rs4601580.

In a particular embodiment, the present invention relates to a method for predicting a response to Tocilizumab comprising analysis of the IL6R SNP polymorphism rs2228145 or rs4129267 in combination with rs6684439.

In a particular embodiment, the present invention relates to a method for predicting a response to Tocilizumab comprising analysis of the IL6R SNP polymorphism rs2228145 or rs4129267 in combination with rs4845618.

In a particular embodiment, the present invention relates to a method for predicting a response to Tocilizumab comprising analysis of the IL6R SNP polymorphism rs2228145 or rs4129267 in combination with rs6427658.

In a particular embodiment, the present invention relates to a method for predicting a response to Tocilizumab comprising analysis of the IL6R SNP polymorphism rs2228145 or rs4129267 in combination with rs8192282.

In a particular embodiment, the present invention relates to a method for predicting a response to Tocilizumab comprising analysis of the IL6R SNP polymorphism rs2228145 or rs4129267 in combination with rs11557725.

In a particular embodiment, the present invention relates to a method for predicting a response to Tocilizumab comprising analysis of the IL6R SNP polymorphism rs2228145 or rs4129267 in combination with rs6694817.

In a particular embodiment, the present invention relates to a method for predicting a response to Tocilizumab comprising analysis of the IL6R SNP polymorphism rs2228145 or rs4129267 in combination with rs11557724.

In a particular embodiment, the present invention relates to a method for predicting a response to Tocilizumab comprising analysis of the IL6R SNP polymorphism rs2228145 or rs4129267 in combination with rs28730733.

In a particular embodiment, the present invention relates to a method for predicting a response to Tocilizumab comprising analysis of the IL6R SNP polymorphism rs2228145 or rs4129267 in combination with rs7549250.

In a particular embodiment, the present invention relates to a method for predicting a response to Tocilizumab comprising analysis of the IL6R SNP polymorphism rs2228145 or rs4129267 in combination with rs7553796.

In a particular embodiment, the present invention relates to a method for predicting a response to Tocilizumab comprising analysis of the IL6R SNP polymorphism rs2228145 or rs4129267 in combination with rs7518199.

In a particular embodiment, the present invention relates to a method for predicting a response to Tocilizumab comprising analysis of the IL6R SNP polymorphism rs2228145 or rs4129267 in combination with rs4845371.

In a particular embodiment, the present invention relates to a method for predicting a response to Tocilizumab comprising analysis of the IL6R SNP polymorphism rs2228145 or rs4129267 in combination with rs6667434.

In a particular embodiment, the present invention relates to a method for predicting a response to Tocilizumab comprising analysis of the IL6R SNP polymorphism rs2228145 or rs4129267 in combination with rs4553185.

In a particular embodiment, the present invention relates to a method for predicting a response to Tocilizumab comprising analysis of the IL6R SNP polymorphism rs2228145 or rs4129267 in combination with rs4845622.

In a particular embodiment, the present invention relates to a method for predicting a response to Tocilizumab comprising analysis of the IL6R SNP polymorphism rs2228145 or rs4129267 in combination with rs4393147.

In a particular embodiment, the present invention relates to a method for predicting a response to Tocilizumab comprising analysis of the IL6R SNP polymorphism rs2228145 or rs4129267 in combination with rs4453032.

In a particular embodiment, the present invention relates to a method for predicting a response to Tocilizumab comprising analysis of the IL6R SNP polymorphism rs2228145 or rs4129267 in combination with rs6664201.

In a particular embodiment, the present invention relates to a method for predicting a response to Tocilizumab comprising analysis of the IL6R SNP polymorphism rs2228145 or rs4129267 in combination with rs4845623.

In a particular embodiment, the present invention relates to a method for predicting a response to Tocilizumab comprising analysis of the IL6R SNP polymorphism rs2228145 or rs4129267 in combination with rs4537545.

In a particular embodiment, the present invention relates to a method for predicting a response to Tocilizumab comprising analysis of the IL6R SNP polymorphism rs2228145 or rs4129267 in combination with rs7529229.

In a particular embodiment, the present invention relates to a method for predicting a response to Tocilizumab comprising analysis of the IL6R SNP polymorphism rs2228145 or rs4129267 in combination with rs4845625.

In a particular embodiment, the present invention relates to a method for predicting a response to Tocilizumab comprising analysis of the IL6R SNP polymorphism rs2228145 or rs4129267 in combination with rs4845626.

In a particular embodiment, the present invention relates to a method for predicting a response to Tocilizumab comprising analysis of the IL6R SNP polymorphism rs2228145 or rs4129267 in combination with rs6689393.

In a particular embodiment, the present invention relates to a method for predicting a response to Tocilizumab comprising analysis of the IL6R SNP polymorphism rs2228145 or rs4129267 in combination with rs4845374.

In a particular embodiment, the present invention relates to a method for predicting a response to Tocilizumab comprising analysis of the IL6R SNP polymorphism rs2228145 or rs4129267 in combination with rs28730735.

In a particular embodiment, the present invention relates to a method for predicting a response to Tocilizumab comprising analysis of the IL6R SNP polymorphism rs2228145 or rs4129267 in combination with rs28730736.

In a particular embodiment, the present invention relates to a method for predicting a response to Tocilizumab comprising analysis of the IL6R SNP polymorphism rs2228145 or rs4129267 in combination with rs11265618.

In a particular embodiment, the present invention relates to a method for predicting a response to Tocilizumab comprising analysis of the IL6R SNP polymorphism rs2228145 or rs4129267 in combination with rs12125954.

In a particular embodiment, the present invention relates to a method for predicting a response to Tocilizumab comprising analysis of the IL6R SNP polymorphism rs2228145 or rs4129267 in combination with rs10159236.

In a particular embodiment, the present invention relates to a method for predicting a response to Tocilizumab comprising analysis of the IL6R SNP polymorphism rs2228145 or rs4129267 in combination with rs10752641.

In a particular embodiment, the present invention relates to a method for predicting a response to Tocilizumab comprising analysis of the IL6R SNP polymorphism rs2228145 or rs4129267 in combination with rs4329505.

In a particular embodiment, the present invention relates to a method for predicting a response to Tocilizumab comprising analysis of the IL6R SNP polymorphism rs2228145 or rs4129267 in combination with rs4240872.

In a particular embodiment, the present invention relates to a method for predicting a response to Tocilizumab comprising analysis of the IL6R SNP polymorphism rs2228145 or rs4129267 in combination with rs4509570.

In a particular embodiment, the present invention relates to a method for predicting a response to Tocilizumab comprising analysis of the IL6R SNP polymorphism rs2228145 or rs4129267 in combination with rs4341355.

In a particular embodiment, the present invention relates to a method for predicting a response to Tocilizumab comprising analysis of the IL6R SNP polymorphism rs2228145 or rs4129267 in combination with rs2229238.

In a particular embodiment, the present invention relates to a method for predicting a response to Tocilizumab comprising analysis of the IL6R SNP polymorphism rs2228145 or rs4129267 in combination with rs7514452.

In a particular embodiment, the present invention relates to a method for predicting a response to Tocilizumab comprising analysis of the IL6R SNP polymorphism rs2228145 or rs4129267 in combination with rs4072391.

In a particular embodiment, the present invention relates to a method for predicting a response to Tocilizumab comprising analysis of the IL6R SNP polymorphism rs2228145 or rs4129267 in combination with rs4379670.

In a particular embodiment, the present invention relates to a method for predicting a response to Tocilizumab comprising analysis of the IL6R SNP polymorphism rs2228145 or rs4129267 in combination with rs11265621.

In a particular embodiment, the present invention relates to a method for predicting a response to Tocilizumab comprising analysis of the IL6R SNP polymorphism rs2228145 or rs4129267 in combination with rs7526293.

In a particular embodiment, the present invention relates to a method for predicting a response to Tocilizumab comprising analysis of the IL6R SNP polymorphism rs2228145 or rs4129267 in combination with rs41310931.

rs952146

In one embodiment, the present invention relates to a method for predicting a response to Tocilizumab comprising analysis of the IL6R SNP polymorphism rs952146 optionally in combination with at least one other genetic variation in the IL6R gene, such as another IL6R SNP.

In one embodiment, the present invention relates to a method for predicting a response to Tocilizumab treatment comprising analysis of the IL6R SNP polymorphism rs952146 alone or in combination with one or more of rs12083537, rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In one embodiment, the present invention does not relate to a method for predicting a response to Tocilizumab treatment comprising or consisting of the analysis of the IL6R SNP polymorphism rs952146.

rs7526293

In one embodiment, the present invention relates to a method for predicting a response to Tocilizumab comprising analysis of the IL6R SNP polymorphism rs7526293 optionally in combination with at least one other genetic variation in the IL6R gene, such as another IL6R SNP.

In one embodiment, the present invention relates to a method for predicting a response to Tocilizumab treatment comprising analysis of the IL6R SNP polymorphism rs7526293 alone or in combination with one or more of rs12083537, rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In a particular embodiment, the present invention relates to a method for predicting a response to Tocilizumab comprising analysis of the IL6R SNP polymorphism rs7526293 in combination with rs4509570.

In a particular embodiment, the present invention relates to a method for predicting a response to Tocilizumab comprising analysis of the IL6R SNP polymorphism rs7526293 in combination with rs12083537.

In a particular embodiment, the present invention relates to a method for predicting a response to Tocilizumab comprising analysis of the IL6R SNP polymorphism rs7526293 in combination with rs952146.

In a particular embodiment, the present invention relates to a method for predicting a response to Tocilizumab comprising analysis of the IL6R SNP polymorphism rs7526293 in combination with rs2228145 or rs4129267.

rs4509570

In one embodiment, the present invention relates to a method for predicting a response to Tocilizumab comprising analysis of the IL6R SNP polymorphism rs7526293 optionally in combination with at least one other genetic variation in the IL6R gene, such as another IL6R SNP.

In one embodiment, the present invention relates to a method for predicting a response to Tocilizumab treatment comprising analysis of the IL6R SNP polymorphism rs4509570 alone or in combination with one or more of rs12083537, rs952146, rs17654071, rs6427631, rs2054855, rs4845615, rs1552481, rs4845617, rs746475, rs6427641, rs11265610, rs12083537, rs1386821, rs7411976, rs4075016, rs4075015, rs12090237, rs4601580, rs6684439, rs4845618, rs6427658, rs8192282, rs11557725, rs6694817, rs11557724, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4509570, rs4341355, rs2229238, rs7514452, rs4072391, rs4379670, rs11265621, and rs7526293. The group may additionally include rs41310931.

In a particular embodiment, the present invention relates to a method for predicting a response to Tocilizumab comprising analysis of the IL6R SNP polymorphism rs7526293 in combination with rs4509570.

In a particular embodiment, the present invention relates to a method for predicting a response to Tocilizumab comprising analysis of the IL6R SNP polymorphism rs4509570 in combination with rs12083537.

In a particular embodiment, the present invention relates to a method for predicting a response to Tocilizumab comprising analysis of the IL6R SNP polymorphism rs4509570 in combination with rs952146.

In a particular embodiment, the present invention relates to a method for predicting a response to Tocilizumab comprising analysis of the IL6R SNP polymorphism rs4509570 in combination with rs2228145 or rs4129267.

rs4075015

In one embodiment, the present invention relates to a method for predicting a response to Tocilizumab comprising analysis of the IL6R SNP polymorphism rs4075015 in combination with at least one other genetic variation in the IL6R gene, such as another IL6R SNP.

In a particular embodiment, the present invention relates to a method for predicting a response to Tocilizumab comprising analysis of the IL6R SNP polymorphism rs4075015 in combination with rs17654071.

In a particular embodiment, the present invention relates to a method for predicting a response to Tocilizumab comprising analysis of the IL6R SNP polymorphism rs4075015 in combination with rs6427631.

In a particular embodiment, the present invention relates to a method for predicting a response to Tocilizumab comprising analysis of the IL6R SNP polymorphism rs4075015 in combination with rs2054855.

In a particular embodiment, the present invention relates to a method for predicting a response to Tocilizumab comprising analysis of the IL6R SNP polymorphism rs4075015 in combination with rs4845615.

In a particular embodiment, the present invention relates to a method for predicting a response to Tocilizumab comprising analysis of the IL6R SNP polymorphism rs4075015 in combination with rs1552481.

In a particular embodiment, the present invention relates to a method for predicting a response to Tocilizumab comprising analysis of the IL6R SNP polymorphism rs4075015 in combination with rs4845617.

In a particular embodiment, the present invention relates to a method for predicting a response to Tocilizumab comprising analysis of the IL6R SNP polymorphism rs4075015 in combination with rs746475.

In a particular embodiment, the present invention relates to a method for predicting a response to Tocilizumab comprising analysis of the IL6R SNP polymorphism rs4075015 in combination with rs6427641.

In a particular embodiment, the present invention relates to a method for predicting a response to Tocilizumab comprising analysis of the IL6R SNP polymorphism rs4075015 in combination with rs11265610.

In a particular embodiment, the present invention relates to a method for predicting a response to Tocilizumab comprising analysis of the IL6R SNP polymorphism rs4075015 in combination with rs12083537.

In a particular embodiment, the present invention relates to a method for predicting a response to Tocilizumab comprising analysis of the IL6R SNP polymorphism rs4075015 in combination with rs1386821.

In a particular embodiment, the present invention relates to a method for predicting a response to Tocilizumab comprising analysis of the IL6R SNP polymorphism rs4075015 in combination with rs7411976.

In a particular embodiment, the present invention relates to a method for predicting a response to Tocilizumab comprising analysis of the IL6R SNP polymorphism rs4075015 in combination with rs4075016.

In a particular embodiment, the present invention relates to a method for predicting a response to Tocilizumab comprising analysis of the IL6R SNP polymorphism rs4075015 in combination with rs12090237.

In a particular embodiment, the present invention relates to a method for predicting a response to Tocilizumab comprising analysis of the IL6R SNP polymorphism rs4075015 in combination with rs4601580.

In a particular embodiment, the present invention relates to a method for predicting a response to Tocilizumab comprising analysis of the IL6R SNP polymorphism rs4075015 in combination with rs6684439.

In a particular embodiment, the present invention relates to a method for predicting a response to Tocilizumab comprising analysis of the IL6R SNP polymorphism rs4075015 in combination with rs4845618.

In a particular embodiment, the present invention relates to a method for predicting a response to Tocilizumab comprising analysis of the IL6R SNP polymorphism rs4075015 in combination with rs6427658.

In a particular embodiment, the present invention relates to a method for predicting a response to Tocilizumab comprising analysis of the IL6R SNP polymorphism rs4075015 in combination with rs8192282.

In a particular embodiment, the present invention relates to a method for predicting a response to Tocilizumab comprising analysis of the IL6R SNP polymorphism rs4075015 in combination with rs11557725.

In a particular embodiment, the present invention relates to a method for predicting a response to Tocilizumab comprising analysis of the IL6R SNP polymorphism rs4075015 in combination with rs6694817.

In a particular embodiment, the present invention relates to a method for predicting a response to Tocilizumab comprising analysis of the IL6R SNP polymorphism rs4075015 in combination with rs11557724.

In a particular embodiment, the present invention relates to a method for predicting a response to Tocilizumab comprising analysis of the IL6R SNP polymorphism rs4075015 in combination with rs28730733.

In a particular embodiment, the present invention relates to a method for predicting a response to Tocilizumab comprising analysis of the IL6R SNP polymorphism rs4075015 in combination with rs7549250.

In a particular embodiment, the present invention relates to a method for predicting a response to Tocilizumab comprising analysis of the IL6R SNP polymorphism rs4075015 in combination with rs7553796.

In a particular embodiment, the present invention relates to a method for predicting a response to Tocilizumab comprising analysis of the IL6R SNP polymorphism rs4075015 in combination with rs7518199.

In a particular embodiment, the present invention relates to a method for predicting a response to Tocilizumab comprising analysis of the IL6R SNP polymorphism rs4075015 in combination with rs4845371.

In a particular embodiment, the present invention relates to a method for predicting a response to Tocilizumab comprising analysis of the IL6R SNP polymorphism rs4075015 in combination with rs6667434.

In a particular embodiment, the present invention relates to a method for predicting a response to Tocilizumab comprising analysis of the IL6R SNP polymorphism rs4075015 in combination with rs4553185.

In a particular embodiment, the present invention relates to a method for predicting a response to Tocilizumab comprising analysis of the IL6R SNP polymorphism rs4075015 in combination with rs4845622.

In a particular embodiment, the present invention relates to a method for predicting a response to Tocilizumab comprising analysis of the IL6R SNP polymorphism rs4075015 in combination with rs4393147.

In a particular embodiment, the present invention relates to a method for predicting a response to Tocilizumab comprising analysis of the IL6R SNP polymorphism rs4075015 in combination with rs4453032.

In a particular embodiment, the present invention relates to a method for predicting a response to Tocilizumab comprising analysis of the IL6R SNP polymorphism rs4075015 in combination with rs6664201.

In a particular embodiment, the present invention relates to a method for predicting a response to Tocilizumab comprising analysis of the IL6R SNP polymorphism rs4075015 in combination with rs4845623.

In a particular embodiment, the present invention relates to a method for predicting a response to Tocilizumab comprising analysis of the IL6R SNP polymorphism rs4075015 in combination with rs4537545.

In a particular embodiment, the present invention relates to a method for predicting a response to Tocilizumab comprising analysis of the IL6R SNP polymorphism rs4075015 in combination with rs7529229.

In a particular embodiment, the present invention relates to a method for predicting a response to Tocilizumab comprising analysis of the IL6R SNP polymorphism rs4075015 in combination with rs4845625.

In a particular embodiment, the present invention relates to a method for predicting a response to Tocilizumab comprising analysis of the IL6R SNP polymorphism rs4075015 in combination with rs4845626.

In a particular embodiment, the present invention relates to a method for predicting a response to Tocilizumab comprising analysis of the IL6R SNP polymorphism rs4075015 in combination with rs6689393.

In a particular embodiment, the present invention relates to a method for predicting a response to Tocilizumab comprising analysis of the IL6R SNP polymorphism rs4075015 in combination with rs4845374.

In a particular embodiment, the present invention relates to a method for predicting a response to Tocilizumab comprising analysis of the IL6R SNP polymorphism rs4075015 in combination with rs28730735.

In a particular embodiment, the present invention relates to a method for predicting a response to Tocilizumab comprising analysis of the IL6R SNP polymorphism rs4075015 in combination with rs28730736.

In a particular embodiment, the present invention relates to a method for predicting a response to Tocilizumab comprising analysis of the IL6R SNP polymorphism rs4075015 in combination with rs11265618.

In a particular embodiment, the present invention relates to a method for predicting a response to Tocilizumab comprising analysis of the IL6R SNP polymorphism rs4075015 in combination with rs12125954.

In a particular embodiment, the present invention relates to a method for predicting a response to Tocilizumab comprising analysis of the IL6R SNP polymorphism rs4075015 in combination with rs10159236.

In a particular embodiment, the present invention relates to a method for predicting a response to Tocilizumab comprising analysis of the IL6R SNP polymorphism rs4075015 in combination with rs10752641.

In a particular embodiment, the present invention relates to a method for predicting a response to Tocilizumab comprising analysis of the IL6R SNP polymorphism rs4075015 in combination with rs4329505.

In a particular embodiment, the present invention relates to a method for predicting a response to Tocilizumab comprising analysis of the IL6R SNP polymorphism rs4075015 in combination with rs4240872.

In a particular embodiment, the present invention relates to a method for predicting a response to Tocilizumab comprising analysis of the IL6R SNP polymorphism rs4075015 in combination with rs4509570.

In a particular embodiment, the present invention relates to a method for predicting a response to Tocilizumab comprising analysis of the IL6R SNP polymorphism rs4075015 in combination with rs4341355.

In a particular embodiment, the present invention relates to a method for predicting a response to Tocilizumab comprising analysis of the IL6R SNP polymorphism rs4075015 in combination with rs2229238.

In a particular embodiment, the present invention relates to a method for predicting a response to Tocilizumab comprising analysis of the IL6R SNP polymorphism rs4075015 in combination with rs7514452.

In a particular embodiment, the present invention relates to a method for predicting a response to Tocilizumab comprising analysis of the IL6R SNP polymorphism rs4075015 in combination with rs4072391.

In a particular embodiment, the present invention relates to a method for predicting a response to Tocilizumab comprising analysis of the IL6R SNP polymorphism rs4075015 in combination with rs4379670.

In a particular embodiment, the present invention relates to a method for predicting a response to Tocilizumab comprising analysis of the IL6R SNP polymorphism rs4075015 in combination with rs11265621.

In a particular embodiment, the present invention relates to a method for predicting a response to Tocilizumab comprising analysis of the IL6R SNP polymorphism rs4075015 in combination with rs7526293.

In a particular embodiment, the present invention relates to a method for predicting a response to Tocilizumab comprising analysis of the IL6R SNP polymorphism rs4075015 in combination with rs41310931.

### Other Combinations of Two SNPs

In a particular embodiment, the present invention relates to a method for predicting a response to Tocilizumab comprising analysis of the IL6R SNP polymorphism rs7553796 in combination with rs10159236.

In a particular embodiment, the present invention relates to a method for predicting a response to Tocilizumab comprising analysis of the IL6R SNP polymorphism rs7553796 in combination with rs7526293.

In a particular embodiment, the present invention relates to a method for predicting a response to Tocilizumab comprising analysis of the IL6R SNP polymorphism rs7553796 in combination with rs4509570.

In a particular embodiment, the present invention relates to a method for predicting a response to Tocilizumab comprising analysis of the IL6R SNP polymorphism rs10159236 in combination with rs7526293.

In a particular embodiment, the present invention relates to a method for predicting a response to Tocilizumab comprising analysis of the IL6R SNP polymorphism rs10159236 in combination with rs4509570.

In a particular embodiment, the present invention relates to a method for predicting a response to Tocilizumab comprising analysis of the IL6R SNP polymorphism rs12090237 in combination with IL6R-rs4509570.

rs2228145 in Combination with Two Other SNPs

In a particular embodiment, the present invention relates to a method for predicting a response to Tocilizumab comprising analysis of the IL6R SNP polymorphism rs2228145 in combination with rs952146 and rs4075015.

In a particular embodiment, the present invention relates to a method for predicting a response to Tocilizumab comprising analysis of the IL6R SNP polymorphism rs2228145 in combination with rs952146 and rs7553796.

In a particular embodiment, the present invention relates to a method for predicting a response to Tocilizumab comprising analysis of the IL6R SNP polymorphism rs2228145 in combination with rs4075015 and rs7553796.

In a particular embodiment, the present invention relates to a method for predicting a response to Tocilizumab comprising analysis of the IL6R SNP polymorphism rs2228145 in combination with rs10159236 and rs7553796.

In a particular embodiment, the present invention relates to a method for predicting a response to Tocilizumab comprising analysis of the IL6R SNP polymorphism rs2228145 in combination with rs10159236 and rs952146.

In a particular embodiment, the present invention relates to a method for predicting a response to Tocilizumab comprising analysis of the IL6R SNP polymorphism rs2228145 in combination with rs10159236 and rs4075015.

In a particular embodiment, the present invention relates to a method for predicting a response to Tocilizumab comprising analysis of the IL6R SNP polymorphism rs2228145 in combination with rs12083537 and rs4075015.

In a particular embodiment, the present invention relates to a method for predicting a response to Tocilizumab comprising analysis of the IL6R SNP polymorphism rs2228145 in combination with rs10159236 and rs952146.

In a particular embodiment, the present invention relates to a method for predicting a response to Tocilizumab comprising analysis of the IL6R SNP polymorphism rs2228145 in combination with rs7526293 and rs952146.

In a particular embodiment, the present invention relates to a method for predicting a response to Tocilizumab comprising analysis of the IL6R SNP polymorphism rs2228145 in combination with rs4509570 and rs952146.

In a particular embodiment, the present invention relates to a method for predicting a response to Tocilizumab comprising analysis of the IL6R SNP polymorphism rs2228145 in combination with rs7526293 and rs4075015.

In a particular embodiment, the present invention relates to a method for predicting a response to Tocilizumab comprising analysis of the IL6R SNP polymorphism rs2228145 in combination with rs4509570 and rs4075015.

In a particular embodiment, the present invention relates to a method for predicting a response to Tocilizumab comprising analysis of the IL6R SNP polymorphism rs2228145 in combination with rs12083537 and rs952146.

In a particular embodiment, the present invention relates to a method for predicting a response to Tocilizumab comprising analysis of the IL6R SNP polymorphism rs2228145 in combination with rs12083537 and IL6R-rs4509570.

In a particular embodiment, the present invention relates to a method for predicting a response to Tocilizumab comprising analysis of the IL6R SNP polymorphism rs2228145 in combination with rs12083537 and rs7553796.

In a particular embodiment, the present invention relates to a method for predicting a response to Tocilizumab comprising analysis of the IL6R SNP polymorphism rs2228145 in combination with rs12083537 and rs10159236.

In a particular embodiment, the present invention relates to a method for predicting a response to Tocilizumab comprising analysis of the IL6R SNP polymorphism rs2228145 in combination with rs12083537 and rs7526293.

In a particular embodiment, the present invention relates to a method for predicting a response to Tocilizumab comprising analysis of the IL6R SNP polymorphism rs2228145 in combination with rs4509570 and rs7553796.

In a particular embodiment, the present invention relates to a method for predicting a response to Tocilizumab comprising analysis of the IL6R SNP polymorphism rs2228145 in combination with rs4509570 and rs10159236.

In a particular embodiment, the present invention relates to a method for predicting a response to Tocilizumab comprising analysis of the IL6R SNP polymorphism rs2228145 in combination with rs4509570 and rs7526293.

In a particular embodiment, the present invention relates to a method for predicting a response to Tocilizumab comprising analysis of the IL6R SNP polymorphism rs2228145 in combination with rs10159236 and rs7526293.

In a particular embodiment, the present invention relates to a method for predicting a response to Tocilizumab comprising analysis of the IL6R SNP polymorphism rs2228145 in combination with rs7553796 and rs7526293.

rs4129267 in Combination with Two Other SNPs

In a particular embodiment, the present invention relates to a method for predicting a response to Tocilizumab comprising analysis of the IL6R SNP polymorphism rs4129267 in combination with rs952146 and rs4075015.

In a particular embodiment, the present invention relates to a method for predicting a response to Tocilizumab comprising analysis of the IL6R SNP polymorphism rs4129267 in combination with rs952146 and rs7553796.

In a particular embodiment, the present invention relates to a method for predicting a response to Tocilizumab comprising analysis of the IL6R SNP polymorphism rs4129267 in combination with rs4075015 and rs7553796.

In a particular embodiment, the present invention relates to a method for predicting a response to Tocilizumab comprising analysis of the IL6R SNP polymorphism rs4129267 in combination with rs10159236 and rs7553796.

In a particular embodiment, the present invention relates to a method for predicting a response to Tocilizumab comprising analysis of the IL6R SNP polymorphism rs4129267 in combination with rs10159236 and rs952146.

In a particular embodiment, the present invention relates to a method for predicting a response to Tocilizumab comprising analysis of the IL6R SNP polymorphism rs4129267 in combination with rs10159236 and rs4075015.

In a particular embodiment, the present invention relates to a method for predicting a response to Tocilizumab comprising analysis of the IL6R SNP polymorphism rs4129267 in combination with rs12083537 and rs4075015.

In a particular embodiment, the present invention relates to a method for predicting a response to Tocilizumab comprising analysis of the IL6R SNP polymorphism rs4129267 in combination with rs10159236 and rs952146.

In a particular embodiment, the present invention relates to a method for predicting a response to Tocilizumab comprising analysis of the IL6R SNP polymorphism rs4129267 in combination with rs7526293 and rs952146.

In a particular embodiment, the present invention relates to a method for predicting a response to Tocilizumab comprising analysis of the IL6R SNP polymorphism rs4129267 in combination with rs4509570 and rs952146.

In a particular embodiment, the present invention relates to a method for predicting a response to Tocilizumab comprising analysis of the IL6R SNP polymorphism rs4129267 in combination with rs7526293 and rs4075015.

In a particular embodiment, the present invention relates to a method for predicting a response to Tocilizumab comprising analysis of the IL6R SNP polymorphism rs4129267 in combination with rs4509570 and rs4075015.

In a particular embodiment, the present invention relates to a method for predicting a response to Tocilizumab comprising analysis of the IL6R SNP polymorphism rs4129267 in combination with rs12083537 and rs952146.

In a particular embodiment, the present invention relates to a method for predicting a response to Tocilizumab comprising analysis of the IL6R SNP polymorphism rs4129267 in combination with rs12083537 and IL6R-rs4509570.

In a particular embodiment, the present invention relates to a method for predicting a response to Tocilizumab comprising analysis of the IL6R SNP polymorphism rs4129267 in combination with rs12083537 and rs7553796.

In a particular embodiment, the present invention relates to a method for predicting a response to Tocilizumab comprising analysis of the IL6R SNP polymorphism rs4129267 in combination with rs12083537 and rs10159236.

In a particular embodiment, the present invention relates to a method for predicting a response to Tocilizumab comprising analysis of the IL6R SNP polymorphism rs4129267 in combination with rs12083537 and rs7526293.

In a particular embodiment, the present invention relates to a method for predicting a response to Tocilizumab comprising analysis of the IL6R SNP polymorphism rs4129267 in combination with rs4509570 and rs7553796.

In a particular embodiment, the present invention relates to a method for predicting a response to Tocilizumab comprising analysis of the IL6R SNP polymorphism rs4129267 in combination with rs4509570 and rs10159236.

In a particular embodiment, the present invention relates to a method for predicting a response to Tocilizumab comprising analysis of the IL6R SNP polymorphism rs4129267 in combination with rs4509570 and rs7526293.

In a particular embodiment, the present invention relates to a method for predicting a response to Tocilizumab comprising analysis of the IL6R SNP polymorphism rs4129267 in combination with rs10159236 and rs7526293.

In a particular embodiment, the present invention relates to a method for predicting a response to Tocilizumab comprising analysis of the IL6R SNP polymorphism rs4129267 in combination with rs7553796 and rs7526293.

### Patients

The patients according to the present invention are individuals in need of treatment for IL6R-related disorders, for which an IL6R inhibitor such as TCZ may be used to treat the disorder, hence the method of the present invention may be used to predict the response of any patient, which is considered for treatment with an IL6R inhibitor.

In a particular embodiment the patients are suffering from rheumatoid arthritis.

Patients according to the present invention include a human being, such as a man, a woman, a post-menopausal women, a pregnant woman, a lactating woman, an infant, a child, an adult. The human being may be an individual of any age such as from newborn to 120 years old, for example from 0 to 6 months, such as from 6 to 12 months, for example from 1 to 5 years, such as from 5 to 10 years, for example from 10 to 15 years, such as from 15 to 20 years, for example from 20 to 25 years, such as from 25 to 30 years, for example from 30 to 35 years, such as from 35 to 40 years, for example from 40 to 45 years, such as from 45 to 50 years, for example from 50 to 60 years, such as from 60 to 70 years, for example from 70 to 80 years, such as from 80 to 90 years, for example from 90 to 100 years, such as from 100 to 110 years, for example from 110 to 120 years.

The patient may be an individual of any race such as a Caucasian, a black person, an East Asian person, a person of Mongoloid race, a person of Ethiopian race, a person of Negroid race, a person of American Indian race, or a person of Malayan race.

Genomic DNA is obtained or isolated from a sample obtained from a patient. The sample may be any sample of bodily fluid or a tissue sample, from which genomic DNA may be isolated according to the knowledge of a person of skill.

The sample is preferably a body fluid such as a whole blood sample, however genomic DNA may be isolated from any cells containing genomic DNA. Genomic DNA may thus be isolated from any of the below body fluids:
Aqueous humour and vitreous humour
Breast milk
Cerebrospinal fluid
Cerumen (earwax)
Endolymph and perilymph
Gastric juice
Mucus (including nasal drainage and phlegm)
Peritoneal fluid
Pleural fluid
Saliva
Sebum (skin oil)
Semen
Sweat
Tears
Vaginal secretion
Vomit
Urine

Alternatively, genomic DNA may be isolated from cells obtained from human tissue such as by scraping e.g. the oral cavity, or from the hair roots.

### Analysing Genetic Variations

Genetic variations according to the present invention may be identified or detected by any suitable method known to a person of skill. A suitable method for identifying or detecting genetic variations is by sequencing a part of or all of a gene (full-length sequencing).

Sequencing can be performed in several different ways, the gold-standard currently being the Sanger method using chain-termination chemistry. However, the so-called next generation sequencing methods are evolving quickly and are capable of sequencing very large areas of the genome rapidly. For deletions, insertions, and SNPs, both Sanger sequencing and next-generation sequencing methods can be applied, but while Sanger sequencing is a laborious and time consuming method, next-generation sequencing techniques offer the ability to sequence entire genes in one run making these the preferred choice when trying to discover new polymorphisms. Copy-number variations (CNVs) can comprise copies or deletions of very large segments of the genome for which quantitative methods such as TaqMan, molecular beacons, etc. are well-suited. Depending on their exact sizes, both Sanger sequencing and next-generation sequencing methods can be well-suited for detecting smaller CNVs, microsatellites etc. For already known SNPs, insertions, and deletions, both TaqMan, molecular beacons, RFLP, qPCR, and various bead-based techniques are well-suited.

### SNP Typing Technology

Genotyping of single nucleotide polymorphisms may be performed by any methods known to the person of skill. As a first step, genomic DNA is purified from a sample from a patient and as a second step, the genomic DNA is analyzed to genotype one or more SNPs. With multiplex assays, it is possible to analyze a number of SNPs and patients simultaneously.

A variety of technologies have been developed for SNP analysis. Most methods are amplification based and the SNP is subsequently detected by primer extension, oligonucleotide ligation, or hybridization of a probe to the amplified product.

### Multiplexed Reactions

In a preferred embodiment, the analysis of SNPs occurs concurrently (such as simultaneously within the same experiment/method), i.e. a multiplexed reaction.

Suitably, the method of the present invention may comprise a multiplexed PCR reaction for the co-amplification of said at least two SNPs.

In one embodiment, the method of the present invention comprises the following sequential steps:

i) a multiplexed PCR reaction in which the SNPs are amplified,
ii) an allele-specific primer extension reaction (ASPE) in which label moieties are incorporated into the ASPE-primers which match the genotype of the sample,
iii) isolating the extension reaction products into separate populations of individual SNP amplification products.

In one embodiment, the labeled moiety referred to in step ii) is a biotin label, such as a biotinylated nucleotide. Further alternative labels include phycoerythrin (PE)-labeled moieties (such as nucleotides). Alternatively, one could use a radio-labeled moiety.

In one embodiment, step iii) comprises a hybridization based isolation of individual populations of SNP amplification products, such as bead-array hybridization.

In one embodiment of the prognostic method according to the invention, the heterozygousity of the at least one SNP is determined.

It will be recognized that alternative methods of labeling the multiplex products other than ASPE, such as single base chain extension (SBCE), Oligonucleotide ligation assay (OLA), or alternatively the PCR products may be directly hybridized to (SNP specific) probe-coupled beads based on the presence or absence of the SNP. SBCE differs from ASPE in several ways; the allele-specific primers 3′-ends overlap one of the nucleotides located right next to the SNP-loci on either the 3′—or the 5′-side of the SNP. When an allele-specific primer hybridizes to a SNP-locus the polymerase elongates it incorporating a biotinylated dideoxy-dNTP (ddNTP), this method has the advantage that a single allele-specific primer can be used to detect up to four different alleles at a given locus, the drawback being that the reaction has to be performed in four different tubes corresponding to the four possible nucleotides ddATP, ddCTP, ddGTP and ddTTP.

Oligonucleotide ligation assay (OLA): The OLA-assay is based on the ability of two oligonucleotides, one labeled the other allele-specific, to anneal immediately adjacent to each other on a complementary target DNA molecule. The two oligonucleotides are then joined covalently by the action of a DNA ligase, provided that the nucleotides at the junction are correctly base-paired. In this way only a primer matching the present allele at a polymorphic locus will be joined to the labeled oligonucleotide and hence emit detectable fluorescence.

Probe-bead based assay: In the probe-bead based assay a multiplex PCR is performed on the SNP-sites of interest with at least one of the primers in each primer-pair being labeled. An allele-specific probe overlapping a suitable area of the polymorphic locus is then prepared and coupled covalently to suitable microspheres. With all other than the perfectly matching PCR-product, the probe will form a loop because of the mismatching base-pair in the middle of the probe-PCR product hybridization complex and this significantly decreases the melting temperature of the complex ensuring that only perfectly hybridized oligonucleotides will remain attached to the probe and hence emit detectable fluorescence.

ASPE, SBCE, OLA and the probe-bead based assays are all suited for the Luminex platform, but different solid base supports such as microarray chips or possibly other beads available for FACS-cytometers etc. could easily be substituted for the Luminex platform.

In one embodiment of the present invention, SNP Genotyping may be performed using the Luminex platform. The Luminex fluorescent microsphere sorter can be used for low to medium throughput genotyping projects, capable of analyzing anywhere from one SNP in one individual up to 50 SNPs in unlimited numbers of individuals. This device analyzes beads labelled with varying ratios of two fluorescent dyes, and can resolve 100 different dye sets. The fluorescent microspheres used in the SNP assay are coupled to oligonucleotides (the “TAG sequence”) such that each particular bead's fluorescent address is associated with a known 20 nucleotide sequence. User-provided oligos, comprised of a SNP specific sequence coupled to the complement of the bead-associated oligo sequence, are used in an allele specific extension reaction with biotinylated nucleotides. These products are combined with selected fluorescent bead-oligo combinations, then incubated with strepavidin-phycoerythrin (SA-PE). As a result, SNP allele-specific extension products are labeled with PE. Reaction products are then analyzed on the Luminex instrument, which quantifies the amount of PE signal associated with a particular fluorescent bead address. Each address is linked to a particular oligo which in turn is linked to a particular SNP based on the experimental design. Thus, differences in signal intensity determine which alleles are present in a given input PCR fragment. The assay input material is a PCR fragment encompassing the SNP of interest. Multiple SNPs can also be assayed simultaneously on the same fragment. One consideration is that the 3′ end of the PCR fragment should be far enough away from the SNP such that the allele specific extension product generated later will incorporate enough biotinylated C nucleotide to yield a good signal following incubation with SA-PE.

Users also need to design the allele specific primer extension (ASPE) primers. Some general guidelines for Luminex ASPE primer design are:
ASPE primers should be synthesized for all sequence variants and be from the same DNA strand (per target sequence).
ASPE primers should be matched for melting temperature at 51-56° C.
ASPE primers should extend out to and include the SNP as the 3′ nucleotide.
The primer is synthesized with the TAG sequence incorporated at the 5′ end.

Two ASPE primers are needed in order to resolve each possible allele pair. There is a preferred order for selection of fluorescent microspheres used in the assay; not all of the 100 available dye possibilities are equally detectable, so it is recommended to start with the TAG sequences on the most favorable dye sets and proceed from there.

### Kit

The present invention further relates to a kit for use in a method for predicting the response of a patient to an IL6R inhibitor such as TCZ, said kit comprising a means for detecting at least one genetic variation, such as an IL6R SNP.

The kit may comprise at least one set of allele-specific primers as a means for detecting one or more genetic variations in or close to the IL6R gene, such as one or more IL6R SNPs.

The kit should comprise the reagents necessary for obtaining the genomic genetic code for at least one genetic variation in the IL6R gene. Optionally the kit may further comprise a control sample, such as DNA-samples with known genotypes for the genetic variation. For example, when the kit is for the detection of SNPs, the kit may comprise DNA samples with known genotypes for at least one SNP.

Optionally the kit may further comprise instructions for use, such as described in the Examples.

Optionally the kit may further comprise a piece of software capable of performing the genotype calls.

The kit may comprise at least one PCR-primer set, such as at least one primer set as provided in table 3.

The kit may comprise at least one ASPE-primer set, such as a primer set as provided in table 4.

When the kit is for detecting SNPs, the PCR-primer set consists of at least one forward and at least one reverse primer sequence being capable of mediating the amplification of a sequence of genetic material, such as DNA, containing at least one polymorphic locus, such as at least one SNP, when subjected to an appropriate PCR-thermocycling sequence and in combination with an appropriate PCR-reagent mixture.

The kit may be used to determine whether a particular patient having a disease involving the IL6R, such as RA, will benefit from treatment with an IL6R inhibitor such as TCZ.

### REFERENCE LIST

(1) Emery P, Keystone E, Tony H P, Cantagrel A, van V R, Sanchez A et al. IL-6 receptor inhibition with tocilizumab improves treatment outcomes in patients with rheumatoid arthritis refractory to anti-tumour necrosis factor biologicals: results from a 24-week multicentre randomised placebo-controlled trial. Ann Rheum Dis 2008 November; 67(11):1516-23.
(2) Genovese M C, McKay J D, Nasonov E L, Mysler E F, da Silva N A, Alecock E et al. Interleukin-6 receptor inhibition with tocilizumab reduces disease activity in rheumatoid arthritis with inadequate response to disease-modifying antirheumatic drugs: the tocilizumab in combination with traditional disease-modifying antirheumatic drug therapy study. Arthritis Rheum 2008 October; 58(10):2968-80.
(3) Jones G, Sebba A, Gu J, Lowenstein M B, Calvo A, Gomez-Reino J J et al. Comparison of tocilizumab monotherapy versus methotrexate monotherapy in patients with moderate to severe rheumatoid arthritis: The AMBITION study. Ann Rheum Dis 2009 Mar. 17.
(4) Maini R N, Taylor P C, Szechinski J, Pavelka K, Broll J, Balint G et al. Double-blind randomized controlled clinical trial of the interleukin-6 receptor antagonist, tocilizumab, in European patients with rheumatoid arthritis who had an incomplete response to methotrexate. Arthritis Rheum 2006 September; 54(9):2817-29.
(5) Nishimoto N, Miyasaka N, Yamamoto K, Kawai S, Takeuchi T, Azuma J. Long-term safety and efficacy of tocilizumab, an anti-IL-6 receptor monoclonal antibody, in monotherapy, in patients with rheumatoid arthritis (the STREAM study): evidence of safety and efficacy in a 5-year extension study. Ann Rheum Dis 2009 October; 68(10):1580-4.
(6) Smolen J S, Beaulieu A, Rubbert-Roth A, Ramos-Remus C, Rovensky J, Alecock E et al. Effect of interleukin-6 receptor inhibition with tocilizumab in patients with rheumatoid arthritis (OPTION study): a double-blind, placebo-controlled, randomised trial. Lancet 2008 Mar. 22; 371(9617):987-97.
(7) Kooloos W M, Huizing a T W, Guchelaar H J, Wessels J A. Pharmacogenetics in treatment of rheumatoid arthritis. Curr Pharm Des 2010; 16(2):164-75.
(8) Liu C, Batliwalla F, Li W, Lee A, Roubenoff R, Beckman E et al. Genome-wide association scan identifies candidate polymorphisms associated with differential response to anti-TNF treatment in rheumatoid arthritis. Mol Med 2008 September; 14(9-10):575-81.
(9) Hamid Y H, Urhammer S A, Jensen D P, Glumer C, Borch-Johnsen K, Jorgensen T et al. Variation in the interleukin-6 receptor gene associates with type 2 diabetes in Danish whites. Diabetes 2004 December; 53(12):3342-5.
(10) Esteve E, Villuendas G, Mallolas J, Vendrell J, Lopez-Bermejo A, Rodriguez M et al. Polymorphisms in the interleukin-6 receptor gene are associated with body mass index and with characteristics of the metabolic syndrome. Clin Endocrinol (Oxf) 2006 July; 65(1):88-91.
(11) Hamid Y H, Rose C S, Urhammer S A, Glumer C, Nolsoe R, Kristiansen O P et al. Variations of the interleukin-6 promoter are associated with features of the metabolic syndrome in Caucasian Danes. Diabetologia 2005 February; 48(2):251-60.
(12) Melzer D, Perry J R, Hernandez D, Corsi A M, Stevens K, Rafferty I et al. A genome-wide association study identifies protein quantitative trait loci (pQTLs). PLoS Genet 2008 May; 4(5):e1000072.
(13) Galicia J C, Tai H, Komatsu Y, Shimada Y, Akazawa K, Yoshie H. Polymorphisms in the IL-6 receptor (IL-6R) gene: strong evidence that serum levels of soluble IL-6R are genetically influenced. Genes Immun 2004 September; 5(6):513-6.
(14) Hassan B, Maxwell J R, Hyrich K L, Barton A, Worthington J, Isaacs J D et al. Genotype at the sIL-6R A358C polymorphism does not influence response to anti-TNF therapy in patients with rheumatoid arthritis. Rheumatology (Oxford) 2010 January; 49(1):43-7.
(15) Marinou I, Walters K, Winfield J, Bax D E, Wilson A G. A gain of function polymorphism in the interleukin 6 receptor influences RA susceptibility. Ann Rheum Dis 2010 June; 69(6):1191-4.
(16) Bortolin S, Black M, Modi H, Boszko I, Kobler D, Fieldhouse D et al. Analytical validation of the tag-it high-throughput microsphere-based universal array genotyping platform: application to the multiplex detection of a panel of thrombophilia-associated single-nucleotide polymorphisms. Clin Chem 2004 November; 50(11):2028-36.
(17) Taylor J D, Briley D, Nguyen Q, Long K, lannone M A, Li M S et al. Flow cytometric platform for high-throughput single nucleotide polymorphism analysis. Biotechniques 2001 March; 30(3):661-9.
(18) Ye F, Li M S, Taylor J D, Nguyen Q, Colton H M, Casey W M et al. Fluorescent microsphere-based readout technology for multiplexed human single nucleotide polymorphism analysis and bacterial identification. Hum Mutat 2001 April; 17(4):305-16.
(19) Rozen S, Skaletsky H. Primer3 on the WWW for general users and for biologist programmers. Methods Mol Biol 2000; 132:365-86.
(20) Okazaki M, Yamada Y, Nishimoto N, Yoshizaki K, Mihara M. Characterization of anti-mouse interleukin-6 receptor antibody. Immunol Lett 2002; 84:231-40.
(21) Hansen S, Leslie R G. TGN1412: scrutinizing preclinical trials of antibody-based medicines. Nature 2006; 441:282.
(22) Yawata H, Yasukawa K, Natsuka S, et al. Structure-function analysis of human IL-6 receptor: dissociation of amino acid residues required for IL-6-binding and for IL-6 signal transduction through gp130. EMBO J 1993; 12:1705-12.

### EXAMPLES

### Example 1-21 Rheumatoid arthritis patients

### Materials and Methods:

### Patients

21 patients with diagnosed rheumatoid arthritis, who failed to respond to treatment with DMARD, were included in the study (RA). All were out patients at the Depts. Of Rheumatology at Rigshospitalet and Gentofte Hospital. Rheumatologists have evaluated the degree of tender joints (TJ) and Swollen joints (SJ) out of 28 joints evaluated before and after three months of TCZ therapy.

### Genotyping of Single Nucleotide Polymorphisms in IL-6R

Genomic DNA was purified from whole blood samples using a Maxwell-16 robot (Promega Corporation, WI, USA), and IL-6R genotypes were determined using in-house multiplex bead-based assays.

### Assay Development

Based on procedures previously described, a multiplexed bead-based assays using the Luminex 100IS flow-cytometer platform (Luminex Corporation, Austin, Tex., USA) were developed. Allele-specific primers were labelled in an allele-specific primer extension (ASPE) reaction using polymerase chain reaction (PCR) amplified SNP-sites as their target sequences. The labelled ASPE-primers were subsequently hybridized to MicroPlex-xTAG (Luminex Corporation) beadsets for detection and counting on the flow-cytometer. PCRs and ASPE-reactions as well as bead-hybridizations were performed in 96-well, 0.2 mL PCR-plates on a GeneAmp PCR System 9600 (Perkin Elmer Corporation, Wellesley, Mass., USA).

### PCR and ASPE

Primer sequences for the multiplex PCRs were designed using Primer3. Multiplex PCR was carried out using Qiagen Multiplex Mastermix (Qiagen, Hilden, Germany), with each PCR containing 1× Qiagen Multiplex Mastermix, 0.2 mM of each HPLC-purified PCR-primer (TAG Copenhagen, Copenhagen, Denmark) and 30 ng genomic DNA in a total reaction volume of 10 μL. The reactions were kept at 95° C. for 15 min to activate the polymerase, followed by 40 cycles at 94° C. for 30 sec, 60° C. for 3 min and 72° C. for 90 sec. After a final extension at 68° C. for 15 min, the reactions were frozen at −20° C. until further use in ASPE-reactions. ASPE-primers were also constructed using Primer3 and subsequently appended to MicroPlex-xTAG ‘tags’. Each ASPE reaction contained 0.375 U Platinum Genotype Tsp Polymerase, 20 mM Tris-HCl (pH 8.4), 50 mM KCl, 1.25 mM MgCl2, 5 mM dATP, dGTP and 5 mM biotin-14-dCTP (Invitrogen Corporation, Carlsbad, Calif., USA) and 5 mM biotin-11-dUTP (Yorkshire Bioscience, Heslington, York, UK), 25 nM HPLC-purified ASPE-primer (TAG Copenhagen, Copenhagen, Denmark), and 0.24 PCR-product in a total reaction volume of 5 μL. The reactions were kept at 96° C. for 2 min to activate the polymerase, followed by 30 cycles at 94° C. for 30 sec, 50° C. for 1 min, and 74° C. for 2 min. After this, the samples were frozen at −20° C. until counting by hybridization to MicroPlex-xTAG microspheres.

TABLE 3
Selected PCR-primer sequences.

| | | SEQ |
|---|---|---|
| | | ID |
| Polymorphism | Forward Primer | NO |
| IL-6R-rs2228145 | GCTTTTGACAGCACCAGCTA | 1 |
| IL-6R-rs4129267 | AGCTGCTGGCAGTGTTACAG | 2 |
| IL-6R-rs11557725 | GGGTAGAGCCGGAAGACAA | 3 |
| IL-6R-rs11557724 | CACCTGCAATGCTTTCCTTT | 4 |
| IL-6R-rs28730735 | GTACCACTGCCCACATTCCT | 5 |
| IL-6R-rs28730736 | GTACCACTGCCCACATTCCT | 6 |
| IL-6R-rs7553796 | GGACGAGTGATGGGTAGCAT | 7 |
| IL-6R-rs4845623 | ATTCCCCAAGAATGGGAATC | 8 |
| IL-6R-rs10159236 | TGGGCAGCAGAAGGAGAC | 9 |
| IL-6R-rs7526293 | CCCAGTTTTTGATGACAATCC | 10 |
| IL-6R-rs4509570 | CCAGAGTGGAGCACTGTGAA | 11 |
| IL-6R-rs12090237 | CCACCTTCCAGAAGCACATAA | 12 |
| IL-6R-rs952146 | TTCTCGAGGTTGAAGCAACA | 13 |
| IL-6R-rs12083537 | GGGTAGGGGGTGGTTAGAGA | 14 |
| IL-6R-rs4075015 | CAGTGTGAGGCCTATGCTGA | 15 |
| IL-6R-rs6684439 | CCCTCTCCCTCCTTCCTAGA | 16 |
| IL-6R-rs6427631 | CCACCTCCCAAGTTCAAGTG | 17 |
| IL-6R-rs2054855 | ACAATGGCAGGCAGAGGTAG | 18 |
| IL-6R-rs4601580 | CACAAACGATCCTCCCATCT | 19 |

TABLE 3
Selected PCR-primer sequences.

| | | SEQ |
|---|---|---|
| | | ID |
| Polymorphism | Reverse Primer | NO |
| IL-6R-rs2228145 | GGACCCATCTCACCTCAGAA | 20 |
| IL-6R-rs4129267 | CTCCACACACGTTCCCTCTT | 21 |
| IL-6R-rs11557725 | CACAACTCACCATCCACCAG | 22 |
| IL-6R-rs11557724 | AGCCAACATGCACATAGCTC | 23 |
| IL-6R-rs28730735 | AAAACACGGCTTGGCATAAT | 24 |
| IL-6R-rs28730736 | AAAACACGGCTTGGCATAAT | 25 |
| IL-6R-rs7553796 | TCCAAGATTTCGGCTACCTG | 26 |
| IL-6R-rs4845623 | CCGGAAATGTGTAACCAAGG | 27 |
| IL-6R-rs10159236 | GGGACTCTCAGGGTGTGTGT | 28 |
| IL-6R-rs7526293 | TCCCATGATGCAGTGAGCTA | 29 |
| IL-6R-rs4509570 | TCAACTGCCTTCCTGTTCCT | 30 |
| IL-6R-rs12090237 | CTGCTCATTTTGGTGGATGA | 31 |
| IL-6R-rs952146 | GCAACTTTCCCTGTGCTACC | 32 |
| IL-6R-rs12083537 | GGGACCAGGATTCAGGAGAC | 33 |
| IL-6R-rs4075015 | TTCCTTGATGGTGATGTGGA | 34 |
| IL-6R-rs6684439 | AAGTGTCCTCTCCCTACCTCTG | 35 |
| IL-6R-rs6427631 | TTTTCAGCACCATAGGAAAGG | 36 |
| IL-6R-rs2054855 | TTGTGACCAGAAATGTCGTAGA | 37 |
| IL-6R-rs4601580 | AACTGAGATTGCACCATTGC | 38 |

TABLE 4
Selected ASPE-primer sequences.

| | | MicroPlex- | | SEQ |
|---|---|---|---|---|
| | | xTAG- | | ID |
| SNP | Allele | beadset | ASPE-primer sequence (‘tagged’) | NO |
| IL-6R | 1 | LUA-17 | CTTTAATCCTTTATCACTTTATCAGGGCAGTGGTACTGAAGAAGAAT | 39 |
| rs2228145 | 2 | LUA-25 | CTTTTCAATTACTTCAAATCTTCAGGGCAGTGGTACTGAAGAAGAAG | 40 |
| IL-6R | 1 | LUA-2 | CTTTATCAATACATACTACAATCACTTGGAGTGGGGTCAATTCTC | 41 |
| rs4129267 | 2 | LUA-96 | ATACTAACTCAACTAACTTTAAACCTTGGAGTGGGGTCAATTCTT | 42 |
| IL-6R | 1 | LUA-30 | TTACCTTTATACCTTTCTTTTTACCACCGACCTCAGCAGCA | 43 |
| rs11557725 | 2 | LUA-47 | CTTCTCATTAACTTACTTCATAATCACCGACCTCAGCAGCC | 44 |
| IL-6R | 1 | LUA-35 | CAATTTCATCATTCATTCATTTCAATGGGGTGCTCCGAGG | 45 |
| rs11557724 | 2 | LUA-9 | TAATCTTCTATATCAACATCTTACGATGGGGTGCTCCGAGA | 46 |
| IL-6R | 1 | LUA-75 | AATCATACCTTTCAATCTTTTACACAATGGCAATGCAGAGGAG | 47 |
| rs28730735 | 2 | LUA-94 | CTTTCTATCTTTCTACTCAATAATCAATGGCAATGCAGAGGAA | 48 |
| IL-6R | 1 | LUA-15 | ATACTTCATTCATTCATCAATTCAGGACCCATCTCACCTCAGAAC | 49 |
| rs28730736 | 2 | LUA-99 | AATCTACACTAACAATTTCATAACGGACCCATCTCACCTCAGAAT | 50 |
| IL-6R | 1 | LUA-43 | CTTTCAATTACAATACTCATTACACAAAAATATTAAATGGGAAATTCCAG | 51 |
| rs7553796 | 2 | LUA-11 | TACAAATCATCAATCACTTTAATCCAAAAATATTAAATGGGAAATTCCAT | 52 |
| IL-6R | 1 | LUA-73 | ATCAAATCTCATCAATTCAACAATAAACAAACTCCTCCAGAATGGAT | 53 |
| rs4845623 | 2 | LUA-7 | CAATTCATTTACCAATTTACCAATAAACAAACTCCTCCAGAATGGAC | 54 |
| IL-6R | 1 | LUA-100 | CTATCTTTAAACTACAAATCTAACGGAGTTTTTGGTTTGATTCTTGG | 55 |
| rs10159236 | 2 | LUA-57 | CAATATCATCATCTTTATCATTACAGGAGTTTTTGGTTTGATTCTTGT | 56 |
| IL-6R | 1 | LUA-28 | CTACAAACAAACAAACATTATCAAACCCTTAATTTAATAATGCATACACCTG | 57 |
| rs7526293 | 2 | LUA-98 | AATCATACTCAACTAATCATTCAACACCCTTAATTTAATAATGCATACACCTA | 58 |
| IL-6R | 1 | LUA-70 | ATACCAATAATCCAATTCATATCAGAGTTCCCACAGACTCAACTGTG | 59 |
| rs4509570 | 2 | LUA-48 | AAACAAACTTCACATCTCAATAATGAGTTCCCACAGACTCAACTGTC | 60 |
| IL-6R | 1 | LUA-18 | TCAAAATCTCAAATACTCAAATCAAGTGCATGGTGTCAGGACC | 61 |
| rs12090237 | 2 | LUA-62 | TCAATCATAATCTCATAATCCAATCAGTGCATGGTGTCAGGACT | 62 |
| IL-6R | 1 | LUA-21 | AATCCTTTCTTTAATCTCAAATCAACATGAGCAAATGGTGGGA | 63 |
| rs952146 | 2 | LUA-58 | CTACTAATTCATTAACATTACTACACATGAGCAAATGGTGGGG | 64 |
| IL-6R | 1 | LUA-76 | AATCTAACAAACTCATCTAAATACGTGAGGGGAGTATTTGTTTACCCT | 65 |
| rs12083537 | 2 | LUA-84 | TCAACTAACTAATCATCTATCAATGAGGGGAGTATTTGTTTACCCC | 66 |
| IL-6R | 1 | LUA-34 | TCATTCATATACATACCAATTCATGGGTGGGGATGGCTCT | 67 |
| rs4075015 | 2 | LUA-52 | TCAATCATCTTTATACTTCACAATGGGTGGGGATGGCTCA | 68 |
| IL-6R | 1 | LUA-13 | CAATAAACTATACTTCTTCACTAATCCAAATGATGGTGTTAGAGGTG | 69 |
| rs6684439 | 2 | LUA-78 | CTATCTATCTAACTATCTATATCATCCAAATGATGGTGTTAGAGGTA | 70 |
| IL-6R | 1 | LUA-14 | CTACTATACATCTTACTATACTTTTAATTTTTGTAATTTTAGTAGCAATGAGGTT | 71 |
| rs6427631 | 2 | LUA-16 | AATCAATCTTCATTCAAATCATCATAATTTTTGTAATTTTAGTAGCAATGAGGTC | 72 |
| IL-6R | 1 | LUA-97 | AATCTCATAATCTACATACACTATGTACTAGCTGACTTAACAGTTTCAGTCG | 73 |
| rs2054855 | 2 | LUA-23 | TTCAATCATTCAAATCTCAACTTTTGTACTAGCTGACTTAACAGTTTCAGTCA | 74 |
| IL-6R | 1 | LUA-24 | TCAATTACCTTTTCAATACAATACTGGTGACTCTCAATTCTTTTTTTTTT | 75 |
| rs4601580 | 2 | LUA-22 | AATCCTTTTTACTCAATTCAATCATGGTGACTCTCAATTCTTTTTTTTTA | 76 |

### Counting by MicroPlex-xTAG Beadsets

Approximately 250 of each of the appropriate anti-tag-coupled MicroPlex-xTAG microspheres were mixed, isolated by centrifugation and resuspended in 1.1× tetramethylammonium chloride (TMAC) buffer (3.3 M TMAC, 0.11% sarkosyl, 55 mM Tris-HCI, 4.4 mM EDTA) (Sigma-Aldrich, St. Louis, Mo., USA). The microsphere suspension, 45 μL, was added to 54 of ASPE-product and the samples were hybridized by heating them to 96° C. for 2 min, followed by 37° C. for 60 min. The microspheres were then washed once in 1004 refrigerator-cold 1×SSPET (0.2 M phosphate buffer, pH 7.4, 2.98 M NaCl, 0.02 M EDTA, 0.01% Triton X-100) (Sigma-Aldrich), resuspended in 70 μL reporter solution containing 8 mg/mL SA-PE (ProZyme, San Leandro, Calif., USA) in 1.0×TMAC buffer, and incubated for 15 min at 37° C. before being analyzed on the Luminex 1001S flow-cytometer.

### Controls

Positive controls obtained from the Coriell Cell Repository (Camden, N.J., USA) with known sequences representing the majority of possible genotypes, as well as no-template PCR negative controls were included in all assay runs. If any of the positive controls produced incorrect genotypes, or if above-background signals were detected in the negative control, the entire plate was discarded and re-run. After each plate run, at least five randomly chosen samples were re-typed on a new plate as an additional control of consistency of the genotypes obtained from the first run.

### Genotype Calls

Genotype calls were performed for each SNP on each plate individually, using the median fluorescence intensity (MFI)-values of the positive controls as guideline for threshold adjustments. For each SNP, the ratios of the MFIs for the alleles were calculated as: Allele A ratio=MFI_{allele A}/(MFI_{allele A}+MFI_{allele B}). If the allelic ratio for one allele was high (usually above 0.80), the sample was considered a homozygote for that allele. If the allelic ratios were approximately equal (usually between 0.30 and 0.70), the sample was considered a heterozygote. If one or more genotypes for a sample were indistinguishable, or if one or more of the SNP genotype calls for a given sample failed due to other reasons, all genotypes for that particular sample were discarded and the sample was re-run. If a sample failed on three separate occasions, the sample was discarded and excluded from further analysis.

### Results

Our preliminary study cohort consisted of 21 TCZ treated rheumatoid arthritis patients genotyped for 12 SNPs in IL-6R. The results of the study are shown in table 5 below.

In this group, we identified one patient with complete lack of effect of Tocilizumab (patient no. 21), as determined from the need to stop therapy and implement alternative treatment with other biologicals. Two other patients showed reduced effect of Tocilizumab treatment (patients no. 7 and 11). All three patients were homozygous for the rare alleles of rs2228145 (corresponding to the Asp358Ala substitution) and rs4129267. None of the patients with good effect of Tocilizumab treatment carried these variants (p<0.0008). There was no apparent differences between the response of patient homozygous for the wild-type (N=7) and patients heterozygous for the rare alleles of rs2228145 (N=11).

The true non-responder (patient no. 21) varied from the two partial responders by being homozygous for the A-variants of rs952146 and the rs4075015 SNPs. Moreover, the rs12083537 polymorphism differentiates between patient 11 and patients 7 and 21 and may therefore provide a further level of differentiation for TCZ outcome.

TABLE 5
IL6-receptor genotypes in 21 TCZ-treated rheumatoid arthritis patients.

| Patient | | | | | | |
|---|---|---|---|---|---|---|
| No. | IL6R-rs2228145 | IL6R-rs4129267 | IL6R-rs11557724 | IL6R-rs28730736 | IL6R-rs7553796 | IL6R-rs10159236 |
| 1 | A/C | C/T | C/C | G/G | C/A | C/C |
| 2 | A/C | C/T | C/C | G/G | C/A | C/C |
| 3 | A/A | C/C | C/C | G/G | C/A | C/A |
| 4 | A/C | C/T | C/C | G/G | C/A | C/C |
| 5 | A/A | C/C | C/C | G/G | C/C | C/A |
| 6 | A/C | C/T | C/C | G/G | C/A | C/C |
| 7 | C/C | T/T | C/C | G/G | C/C | C/C |
| 8 | A/C | C/T | C/C | G/G | C/C | C/C |
| 9 | A/C | C/T | C/C | G/G | C/C | C/A |
| 10 | A/A | C/C | C/C | G/G | A/A | C/C |
| 11 | C/C | T/T | C/C | G/G | C/C | C/C |
| 12 | A/C | C/T | C/C | G/G | C/A | C/C |
| 13 | A/A | C/C | C/C | G/G | C/C | A/A |
| 14 | A/C | C/T | C/C | G/G | C/A | C/C |
| 15 | A/A | C/C | C/C | G/G | C/A | C/A |
| 16 | A/C | C/T | C/C | G/G | C/C | C/A |
| 17 | A/A | C/C | C/C | G/G | A/A | C/C |
| 18 | A/A | C/C | C/C | G/G | C/A | C/A |
| 19 | A/C | C/T | C/C | G/G | C/C | C/A |
| 20 | A/C | C/T | C/C | G/G | C/C | C/A |
| 21 | C/C | T/T | C/C | G/G | C/C | C/C |
| Patient | | | | | | |
| No. | IL6R-rs7526293 | IL6R-rs4509570 | IL6R-rs12090237 | IL6R-rs952146 | IL6R-rs12083537 | IL6R-rs4075015 |
| 1 | C/C | C/C | G/G | A/A | A/A | A/A |
| 2 | C/T | C/G | G/G | A/A | A/G | A/T |
| 3 | C/C | C/C | G/G | A/G | A/G | A/T |
| 4 | C/C | C/C | G/G | A/G | A/A | A/T |
| 5 | C/T | C/G | G/G | A/G | A/G | A/T |
| 6 | C/C | C/C | G/G | A/A | A/A | T/T |
| 7 | C/C | C/C | G/G | A/G | A/A | A/T |
| 8 | C/T | C/G | G/G | A/A | A/A | A/A |
| 9 | C/C | C/C | G/G | G/G | A/A | T/T |
| 10 | C/T | C/G | G/G | A/A | A/G | A/T |
| 11 | C/C | C/C | G/G | A/G | A/G | A/T |
| 12 | C/T | C/G | G/G | A/G | A/A | A/T |
| 13 | C/C | C/C | G/G | A/G | A/G | A/T |
| 14 | C/C | C/C | G/G | A/G | A/G | A/T |
| 15 | C/T | C/G | G/G | A/A | A/A | A/A |
| 16 | C/C | C/C | G/G | G/G | A/A | T/T |
| 17 | C/T | C/G | G/G | G/G | A/G | T/T |
| 18 | C/T | C/G | G/G | A/A | A/A | A/A |
| 19 | C/C | C/C | G/G | A/G | A/G | T/T |
| 20 | C/C | C/C | G/G | A/G | A/G | A/T |
| 21 | C/C | C/C | G/G | A/A | A/A | A/A |

### Discussion

Our results suggest that both the rs2228145 and the rs4129267 polymorphisms are of value in predicting the clinical response to TCZ. Thus, the one non-responder in this study, and the only two partial responders were all homozygous for the C-allele of rs2228145 and the T-allele of rs4129267. These data suggest, however, that 1) homozygousity for the C-allele is not a sufficient marker for predicting lack of response (since two patients were homozygous and yet exhibited impaired or partial responses), and 2) heterozygousity for the C-allele of rs2228145 is of no predictive value (since 11 heterozygous patients all responded well).

We hypothesize that the rs2228145 (C/C) or the rs4129267 (T/T) polymorphism combined with the rs952146 (A/A) or rs4075015 (A/A) polymorphism are better predictors of the response to TCZ than any of the polymorphisms alone, since the only non-responder in this study had unique combinations of these polymorphisms.

Hasan et al. have previously investigated whether the sIL-6R Asp358Ala polymorphism (corresponding to rs2228145) influenced the response to anti-TNF therapy in patients with rheumatoid arthritis, and showed that it did not. Marinou et al. investigated whether the same polymorphism influenced RA susceptibility and failed to show that it did (p=0.08). They confirmed a gene-dose association between IL-6R A358D genotypes and sIL-6R levels in a healthy British population and found that low sIL-6R levels are a risk factor for the development of RA. Marinou et al recognized, however, that 49% of the variance in sIL-6R levels in European Americans is attributable to the rs8192284 (corresponding to rs2228145) genotype. The authors of both papers put their findings into perspective by suggesting that the Asp358Ala polymorphism may be useful as a marker predicting the response to TCZ. However, neither of the papers present any data supporting this notion. Neither do any of them specify whether the A/A-, C/A- or C/C-genotypes potentially confer a risk for lack of response to TCZ.

Our data show that homozygousity for the C-allele of rs2228145 is a risk factor for predicting poor or no response to TCZ. Moreover, we show that homozygousity for the T-allele of the rs4129267 polymorphism, which is in strong linkage disequilibrium with rs2228145, predicts TCZ response just as well.

However, in contrast to the hypothesis of Hassan et al. and Marinou et al., the present data show that the rs2228145 SNP is unable to distinguish patients which are true non-responders, i.e. patients with a complete lack of response to TCZ with patients exhibiting a partial or decreased response to TCZ. Patients exhibiting a partial response to TCZ may still have some benefit of treatment with TCZ, particularly in cases where no other drugs are useful.

The data show that measurement of either of rs2228145 or rs4129267 in combination with the rs952146, rs4075015 or rs12083537 polymorphisms may be necessary to completely resolve differentiation between patients responding poorly and patients completely lacking effect of TCZ.

The usefulness of the SNPs rs4129267 and rs2228145 may be explained in two, not mutually exclusive, ways: i) it may rely upon an increase in the levels of membrane-bound IL-6-receptor and soluble IL-6-receptor in individuals homozygous for the minor alleles as demonstrated by Marinou et al. which may cause a decrease in the number of receptors being occupied by tocilizumab, or ii) it may rely upon a linkage disequilibrium between rs4129267 and rs2228145, on the one hand, and the DNA sequences encoding residues number residues 252-55 and 297-307, on the other. We hypothesize that SNPs within the latter offer an even better prediction of the effect of tocilizumab than rs4129267 and rs2228145.

Table 6 below shows the clinical data of the patients (samples) from example 1. Rheumatologists have evaluated the degree of tender joints (TJ) and Swollen joints (SJ) before and after three months of TCZ therapy.

A number of the patients were excluded from the study due to another diagnosis.

The test score of tender joints is the number of tender joints out of 28 joints evaluated. TJ is indicated with Arabic numerals in the table.

The test score of swollen joints is likewise the number of swollen joints out of 28 joints evaluated. SJ is indicated with Roman numerals in the table.

Before treatment, patient 21 scored 9/VI and after treatment the score was 6/VI. The TJ score is thus decreased by 33% in response to TCZ treatment and the SJ score is unchanged in response to TCZ treatment. hence, patient 21 is considered a true non-responder as there is less than 30% reduction in disease symptoms in average, cf. table 7.

Patient 7, who is also homozygous for rs2228145 and rs4129267, goes from 16 to 8 tender joints, hence this patient is regarded a partial responder as there is a 50% decrease in disease symptoms

Patient 11 goes from 24 to 14 tender joints, however, her swollen joints disappear completely in response to TCZ treatment. This patient is also regarded as a partial responder as there is a 42% reduction in TJ and a complete recovery from SJ. Patient 11 was previously treated, albeit unsuccessfully, with high doses of prednisolon, rituximab, anti-TNF therapeutics and orencia, so a decrease in disease activity for such a patient is a highly desirable result.

Hence, it is of value that a test for predicting the response to TCZ is capable of distinguishing non-responders from partial responders so that all RA patients who may benefit from TCZ therapy are treated with TCZ.

The clinical data support the hypothesis that the rs2228145- and rs4129267-polymorphisms have a value as predictors for response to TCZ, but are unable to distinguish partial responders from non-responders. A clinical test analyzing only the rs2228145 or the rs4129267 would, in addition to excluding non-responders, lead to exclusion of patients with a partial response to TCZ.

The remainder of the patients has a good effect of TCZ and were considered complete responders, see table 7.

TABLE 6
Clinical data from rheumatoid arthritis
patients treated with Tocilizumab

| Sam- | | Date of | TJ/SJ | TJ/SJ |
|---|---|---|---|---|
| ple # | Initials | birth | before | after |
| 1 | S T | 4 Apr. 1959 | 22/IV | 3/I |
| 2 | S N | 21 Jan. 1937 | 10/X | 0/0 |
| 3 | K S | 14 Feb. 1956 | 11/VII | 2/0 |
| 4 | H T B | 18 Dec. 1976 | 22/XVIII | 0/0 |
| 7 | J B | 1 Jun. 1953 | 16/0 | 8/0 |
| 8 | S E Ø | 18 Jul. 1948 | 10/VIII | 0/0 |
| 10 | E E | 3 May 1953 | 15/VII | 2/0 |
| 11 | K W S S | 17 Apr. 1979 | 24/VIII | 14/0 |
| 12 | R G J | 27 Jul. 1939 | 6/VI | 0/0 |
| 13 | N J | 11 Sep. 1967 | 11/II | 2/0 |
| 14 | J L | 26 Sep. 1954 | 26/VIII | 2/0 |
| 15 | A M K | 1 Feb. 1943 | 18/II | 0/I |
| 16 | B Ø | 28 Sep. 1968 | 8/VI | 2/0 |
| 17 | A R | 29 Sep. 1948 | 6/VI | 0/0 |
| 19 | K M N | 21 Feb. 1987 | 12/VIII | 2-0 |
| 21 | J C H S | 28 Mar. 1959 | 9/VI | 6/VI |

Table 6 shows that the clinical response to TCZ varies from patient to patient. Some patients have a complete response to TCZ, i.e. their joint symptoms disappear completely in response to TCZ therapy and others exhibit a partial or no response to TCZ.

When a response is complete, the disease symptoms are either completely gone or almost completely gone. As a rule of thumb, if the disease symptoms are decreased by more than approximately 90%, the response may be considered complete. Partial response implies that the drug response is not complete. As a rule of thumb, a reduction of disease symptoms by approximately 30% or more on clinical examination or by other means may be considered a partial response. Patients who have no (absent) response are non-responders. As a rule of thumb, non-responders have less than approximately 30% reduction in disease symptoms.

In table 7, the percent reduction of disease symptoms has been calculated for the patients in table 6. Table 7 further indicates whether the patients are considered complete responders, partial responders or non-responders.

TABLE 7
Type of responder

| | | | Reduction | Average | |
|---|---|---|---|---|---|
| Patient | TJ/SJ | TJ/SJ | in % | reduction | Type of |
| # | before | after | (TJ/SJ) | in % | responder |
| 1 | 22/IV | 3/I | 86/75 | 80 | Partial |
| 2 | 10/X | 0/0 | 100/100 | 100 | Complete |
| 3 | 11/VII | 2/0 | 82/100 | 91 | Complete |
| 4 | 22/XVIII | 0/0 | 100/100 | 100 | Complete |
| 7 | 16/0 | 8/0 | 50/— | 50 | Partial |
| 8 | 10/VIII | 0/0 | 100/100 | 100 | Complete |
| 10 | 15/VII | 2/0 | 87/100 | 94 | Complete |
| 11 | 24/VIII | 14/0 | 42/100 | 71 | Partial |
| 12 | 6/VI | 0/0 | 100/100 | 100 | Complete |
| 13 | 11/II | 2/0 | 82/100 | 91 | Complete |
| 14 | 26/VIII | 2/0 | 92/100 | 96 | Complete |
| 15 | 18/II | 0/I | 100/50 | 50 | Complete* |
| 16 | 8/VI | 2/0 | 75/100 | 88 | Complete* |
| 17 | 6/VI | 0/0 | 100/100 | 100 | Complete |
| 19 | 12/VIII | 2-0 | 83/100 | 92 | Complete |
| 21 | 9/VI | 6/VI | 33/0 | 17 | Non |

*Patients 15 and 16 were considered complete responders despite having an average decrease in disease symptoms of less than 90% in average. In particular, patient 16 was considered a complete responder as the average reduction of disease symptoms was very close to 90%. Patient 15 was considered a complete responder due to the fact that there was a 100% decrease in tender joints and the fact that this patient did not suffer much from swollen joints prior to treatment with TCZ.

Although patient 1 exhibits quite a high response to TCZ (80%), the average decrease in disease symptoms is less than 90%, hence patient 1 may be considered a partial responder. Interestingly, this patient does not have the same genotype for rs2228145 and rs4129267 as the other patients exhibiting a decreased (partial or no) response to TCZ, indicating that additional or even alternative IL6R SNPs may be of importance for determining the response to TCZ.

Patient 1 has the same genotype for the SNPs rs952146, rs12083537 and rs4075015 as the non-responding patient 21.

### Materials and Methods:

### Patients

A further 22 patients were included in the study as compared to example 1, i.e. in total 43 patients.

### Methods

CRP was measured at the Depts. Of Clinical Biochemistry at Rigshospitalet and Gentofte Hospital using standard tests. DAS28-CRP was calculated at both time points as 0.56*√(tender joints)+0.28*√/(swollen joints)+0.70*Ln(CRP)+0.014*VAS, where VAS is a Visual Analouge Scale ranging from 0 though 10, on which the patient ranges his or her pain. Further methods are described in detail in Example 1.

### Results

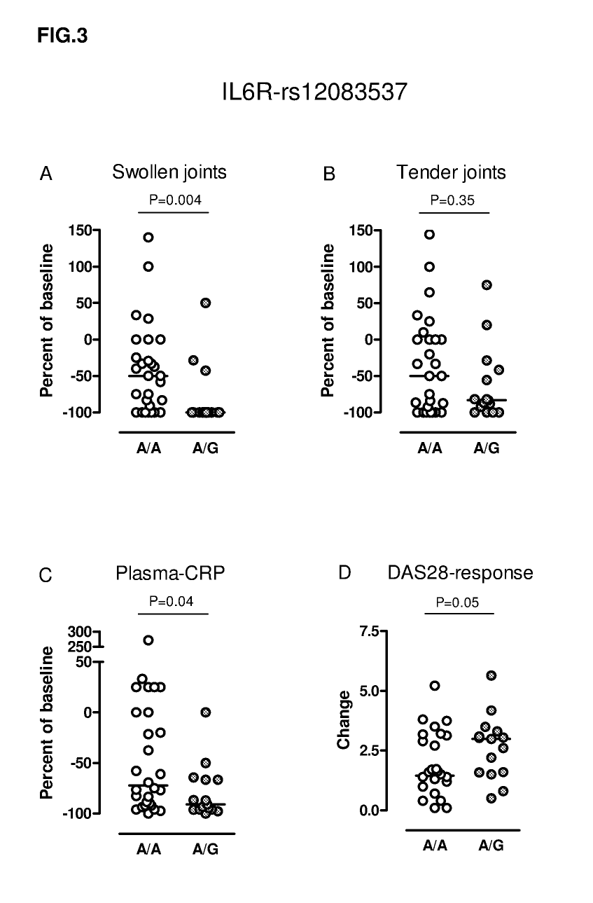

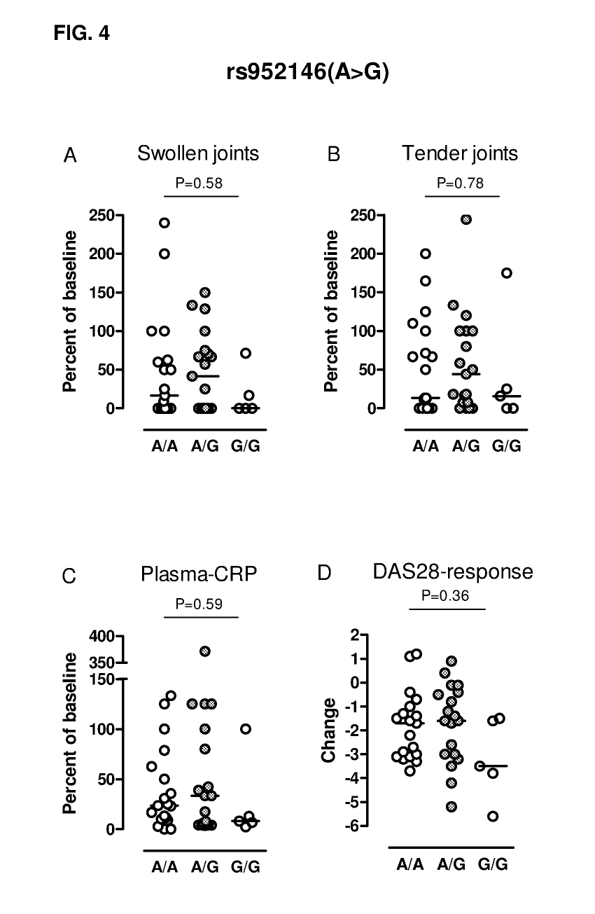

Our study cohort consisted of 43 TCZ treated rheumatoid arthritis patients genotyped for 12 SNPs in IL-6R. The results of the study are shown in FIGS. 3 to 10.

The SNP that most successfully predicted the clinical response was rs12083537 (FIG. 3). While no patients were homozygous for the minor allele, fifteen patients who were heterozygous had a significantly better response to TCZ than the remaining 28 patients who were homozygous for the major allele, as determined by the reduction in swollen joint number (p=0.004, FIG. 3A), circulating CRP levels (p=0.004, FIG. 3C) and change in DAS28 score (p=0.05, FIG. 3D).

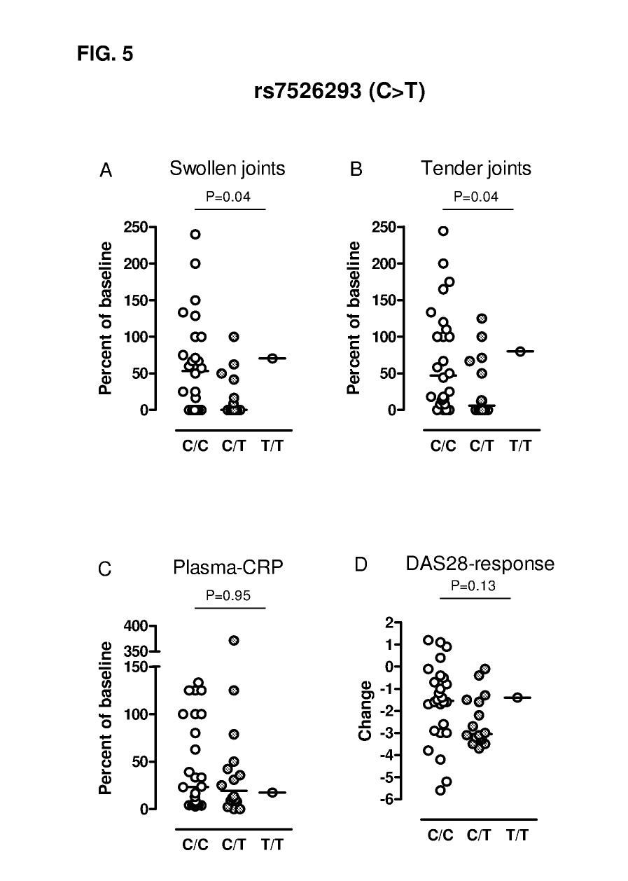

Rs952146, a SNP located in the promotor region of IL-6R upstream for, and in proximity with, rs12083537 did not prove useful in predicting the effect of TCZ. Also the SNP rs7526293 had predictive value. Thus, patients who were heterozygous for this SNP had better effect of TCZ than patients who were homozygous for the major allele, both in terms of reduction in the number of swollen joints (p=0.04, FIG. 5A) and tender joints (p=0.04, FIG. 5B). Upon exclusion of the one patient, who was homozygous for the minor allele, the p-values were reduced to 0.02 and 0.02, respectively. The amelioration of joint symptoms was not, however, reflected in reductions in the level of circulating CRP (FIG. 5C), which prevented the improvement in DAS28 score from becoming significant (p=0.13), since CRP is one of the parameters on the basis of which DAS28 is calculated.

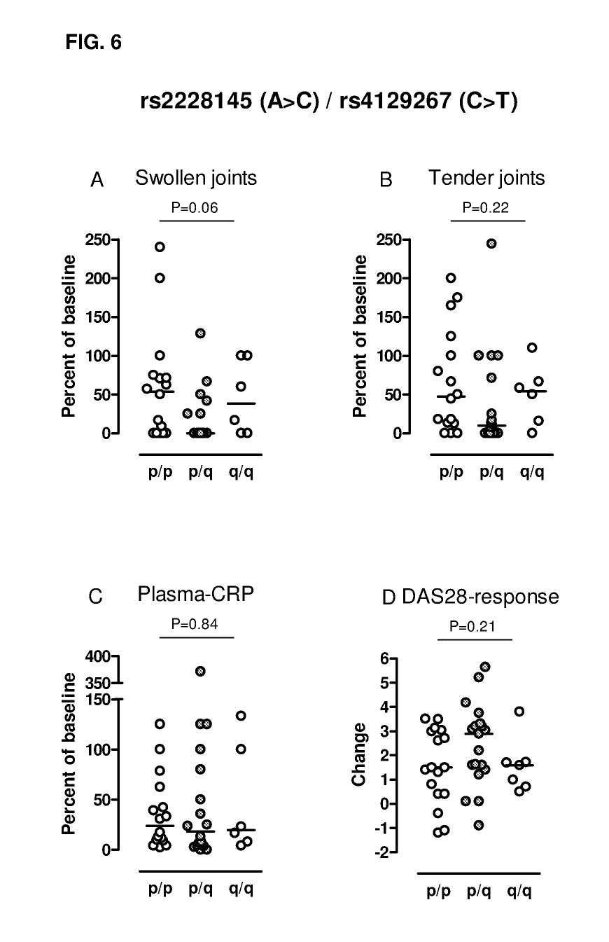

The SNP that has been studied most intensively so far in other connections, rs2228145, was associated, with borderline-significance, with the clinical response to TCZ, as measured by the reduction in swollen joints (FIG. 6A), but with none of the other parameters measured (FIGS. 6B-D). This pattern was mirrored exactly by the rs4129267 polymorphism, which is in strong linkage disequilibrium with rs2228145.

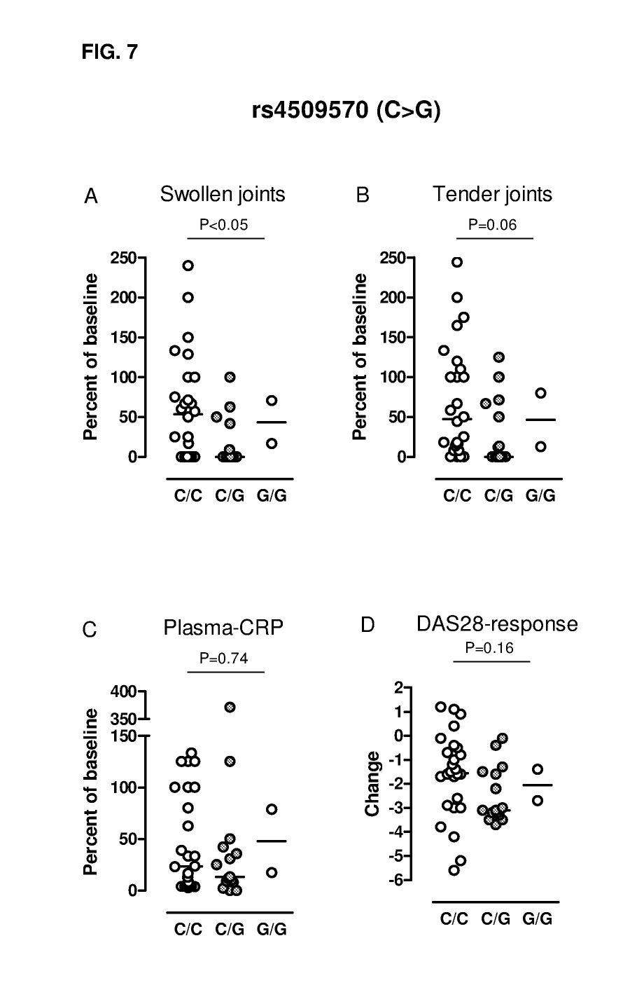

The SNP rs4509570 was strongly linked with rs7526293 mentioned above; the only diversion between them being one patient, who was heterozygous rs7526293 but homozygous for the minor allele of s4509570. Consequently an association between rs4509570 and reduction in the numbers of swollen joints (FIG. 7A), and a tendency association towards an association between rs4509570 and reduction of tender joints (FIG. 7B) were observed.

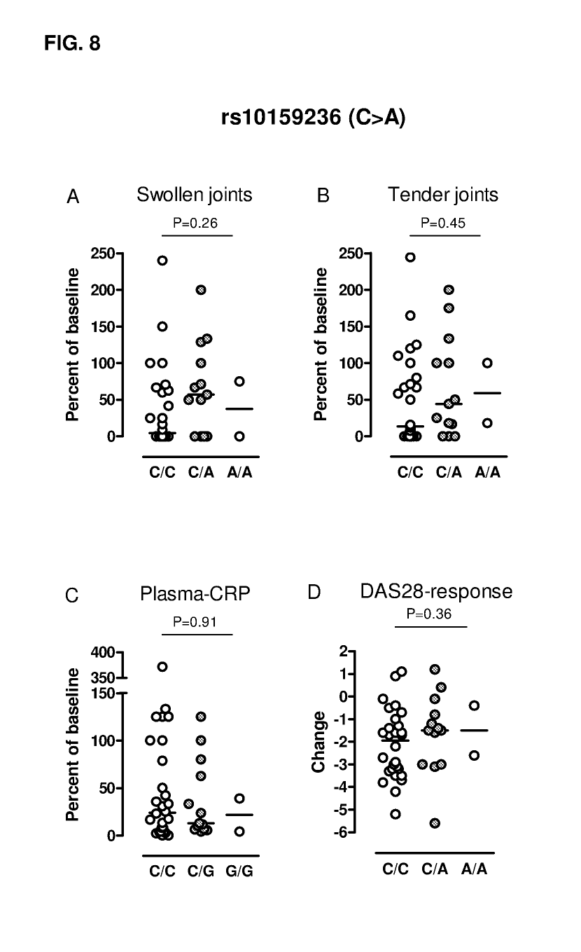

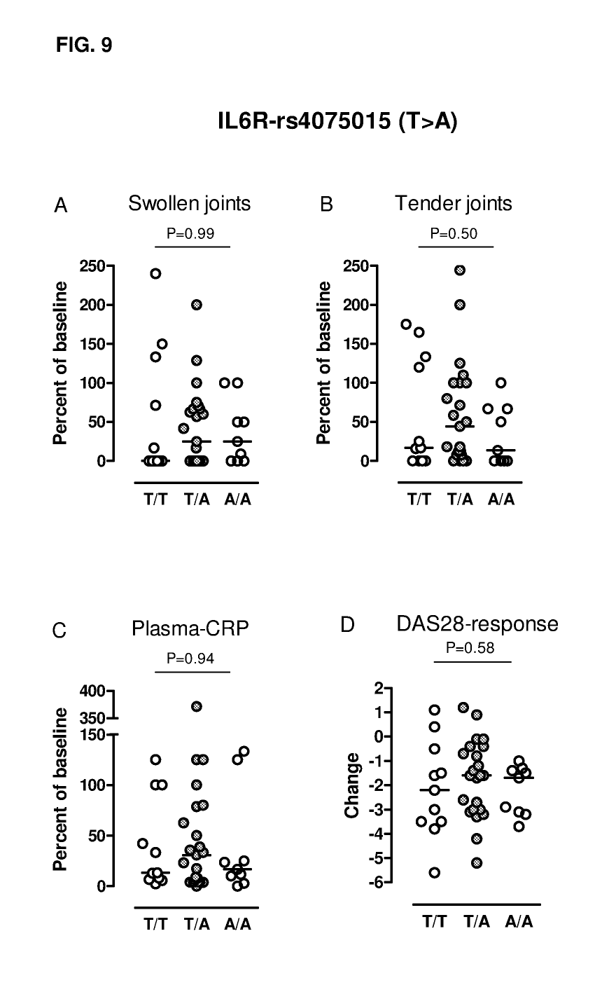

The SNPs rs10159236 and 4075015 individually were not able to predict the clinical response to TCZ therapy (FIGS. 8 and 9) with p-values for swollen joints of 0.26 and 0.99, respectively.

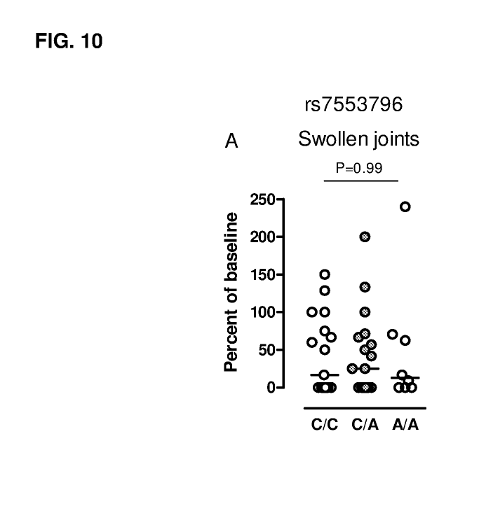

The remaining SNP rs7553796 was of no use as predictor of the clinical response (shown in FIG. 10 for swollen joints only).

In conclusion, the following SNPs were of predictive value when measured individually: rs12083537, rs7526293, rs2228145, rs4129267 and rs4509570.

The following SNPs were not found to be of predictive value in the present study when measured individually: rs952146, rs10159236, rs4075015, rs7553796.

In table 8 is shown the genotypes of the 43 patients of the present study.

TABLE 8
IL6-receptor genotypes in 43 TCZ-treated rheumatoid arthritis patients.

| Patient | IL6R- | IL6R- | IL6R- | IL6R- | IL6R- | IL6R- | IL6R- | IL6R- | IL6R- |
|---|---|---|---|---|---|---|---|---|---|
| No. | rs2228145 | rs4129267 | rs7553796 | rs10159236 | rs7526293 | rs4509570 | rs952146 | rs12083537 | rs4075015 |
| 1 | A/A | C/C | C/A | C/A | C/C | C/C | A/A | A/A | A/T |
| 2 | A/A | C/C | A/A | C/C | C/C | C/C | A/A | A/A | T/T |
| 3 | A/A | C/C | C/A | C/A | C/C | C/C | A/G | A/A | T/T |
| 4 | A/C | C/T | C/C | C/A | C/C | C/C | A/A | A/A | A/A |
| 5 | A/C | C/T | C/A | C/C | C/C | C/C | A/G | A/A | A/T |
| 6 | A/C | C/T | C/C | C/A | C/C | C/C | A/G | A/A | A/T |
| 7 | A/C | C/T | C/C | C/A | C/C | C/C | G/G | A/A | T/T |
| 8 | A/C | C/T | C/A | C/C | C/C | C/C | A/G | A/A | A/T |
| 9 | A/C | C/T | C/A | C/C | C/C | C/C | A/G | A/A | A/T |
| 10 | A/C | C/T | C/A | C/C | C/C | C/C | A/A | A/A | A/A |
| 11 | A/C | C/T | C/C | C/A | C/C | C/C | A/G | A/A | A/T |
| 12 | C/C | T/T | C/C | C/C | C/C | C/C | A/A | A/A | A/A |
| 13 | C/C | T/T | C/C | C/C | C/C | C/C | G/G | A/A | T/T |
| 14 | C/C | T/T | C/C | C/C | C/C | C/C | A/A | A/A | A/T |
| 15 | C/C | T/T | C/C | C/C | C/C | C/C | A/G | A/A | A/T |
| 16 | C/C | T/T | C/C | C/C | C/C | C/C | A/A | A/A | A/A |
| 17 | A/A | C/C | C/A | C/A | C/C | C/C | A/G | A/G | A/T |
| 18 | A/A | C/C | C/C | A/A | C/C | C/C | A/G | A/G | A/T |
| 19 | A/A | C/C | C/A | C/A | C/C | C/C | G/G | A/G | T/T |
| 20 | A/A | C/C | C/A | C/A | C/C | C/C | A/G | A/G | A/T |
| 21 | A/C | C/T | C/A | C/C | C/C | C/C | A/G | A/G | A/T |
| 22 | A/C | C/T | C/C | C/A | C/C | C/C | G/G | A/G | T/T |
| 23 | A/C | C/T | C/C | C/A | C/C | C/C | A/G | A/G | T/T |
| 24 | C/C | T/T | C/C | C/C | C/C | C/C | A/G | A/G | A/T |
| 25 | C/C | T/T | C/C | C/C | C/C | C/C | A/G | A/G | T/T |
| 26 | A/A | C/C | C/C | A/A | C/C | C/C | A/G | A/A | A/T |
| 27 | A/A | C/C | C/A | C/A | C/T | C/G | A/A | A/A | A/A |
| 28 | A/A | C/C | A/A | C/C | C/T | G/G | A/A | A/A | A/A |
| 29 | A/A | C/C | A/A | C/C | C/T | C/G | A/A | A/A | A/T |
| 30 | A/A | C/C | A/A | C/C | C/T | C/G | A/G | A/A | T/T |
| 31 | A/A | C/C | C/A | C/A | C/T | C/G | A/A | A/A | A/A |
| 32 | A/A | C/C | A/A | C/C | C/T | C/G | A/A | A/A | A/A |
| 33 | A/C | C/T | C/C | C/C | C/T | C/G | A/A | A/A | A/A |
| 34 | A/C | C/T | C/A | C/C | C/T | C/G | A/A | A/A | A/A |
| 35 | A/C | C/T | C/A | C/C | C/T | C/G | A/G | A/A | A/T |
| 36 | A/C | C/T | C/A | C/C | C/T | C/G | A/G | A/A | A/T |
| 37 | A/A | C/C | A/A | C/C | C/T | C/G | A/A | A/G | A/T |
| 38 | A/A | C/C | A/A | C/C | C/T | C/G | G/G | A/G | T/T |
| 39 | A/C | C/T | C/C | C/C | C/T | C/G | A/A | A/G | T/T |
| 40 | A/C | C/T | C/A | C/C | C/T | C/G | A/A | A/G | A/T |
| 41 | A/C | C/T | C/A | (C/C) | C/T | C/G | A/A | A/G | A/T |
| 42 | A/C | C/T | C/A | C/C | C/T | C/G | A/A | A/G | A/T |
| 43 | A/A | C/C | A/A | C/C | T/T | G/G | A/G | A/A | A/T |
| | IL6R- | IL6R- | IL6R- | IL6R- | IL6R- | IL6R- | IL6R-rs952146 | IL6R- | IL6R- |
| | rs2228145 | rs4129267 | rs7553796 | rs10159236 | rs7526293 | rs4509570 | | rs12083537 | rs4075015 |

### Discussion

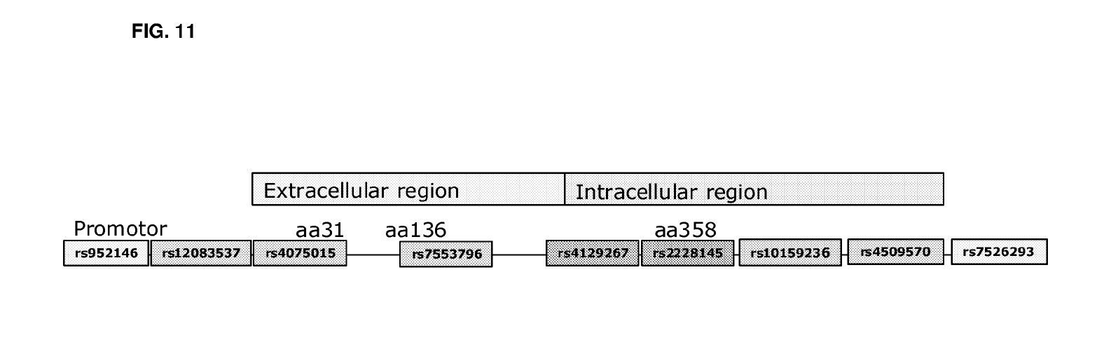

Our results suggest that the SNPs rs12083537, rs7526293, rs4509570 and, to a lesser extent, the strongly coupled SNPs rs2228145 and rs4129267 are of value in predicting the clinical response to TCZ. The SNPs rs952146, rs10159236, rs4075015 and rs7553796 apparently were not useful as predictors of TCZ's effect. The localization of these SNPs within the IL-6R gene is shown in FIG. 11 and Table 2.

The results can be summarized as follows:

| Gene | dbSNP ID | Genotype | Effect |
|---|---|---|---|
| IL6R | rs7526293 | C/T | Predicts responders |
| IL6R | rs12083537 | A/G | Predicts responders |
| IL6R | rs4509570 | C/G | Predicts responders |
| IL6R | rs2228145 | A/C | Predicts responders |
| IL6R | rs4129267 | C/T | Predicts responders |

Rs12083537 is in an intron in proximity to the promotor, and thus does not represent potential binding-sites for TCZ, but is likely to determine the expression of IL6R. Furthermore, it may be linked to an as yet unidentified determinant for TCZ's binding. As a third possibility it may be a potential binding site for miRNA. Apparently, being heterozygous for the rs12083537 polymorphism predicts a good response to TCZ, since the median improvement in the number of swollen joints in this group was 100%. In our cohort, approximately ⅓ of the patients had this genotype (none were homozygotic for the minor allele). We regard swollen joint number as the most useful outcome parameter, since it is more objective than tender joints, and since treatment with TCZ invariably reduces CRP-levels regardless of clinical response. The DAS28-CRP score used here includes both CRP, tender joints and another subjective parameter, the visual analogue score (VAS), and is thus considered less objective than swollen joints assessed by the examining physician.

Intriguingly, patients who were heterozygous for rs2228145 and rs4129267, that are in strong linkage disequilibrium, displayed the best response. Two mutually not exclusive hypotheses may explain this: rs2228145 is a gain-of-function polymorphism of which the minor allele is associated with increased levels of sIL-6R, due to increased proteolytic cleavage (shedding). Patients homozygous for the major allele may represent a disease entity, which is not driven by IL-6 to the same extent as heterozygous patients, or patients who are homozygous for the gain-of-function allele. This would explain the poor effect of TCZ in some patients with the wild type, compared with the effect in heterozygous patients. Another explanation may be that the dosage of TCZ used did not sufficiently inhibit the high sIL-6R levels of at least some patients who homozygous for the minor allele, nor the presumably high expression of membrane-bound IL-6R of patients homozygous for the major allele.

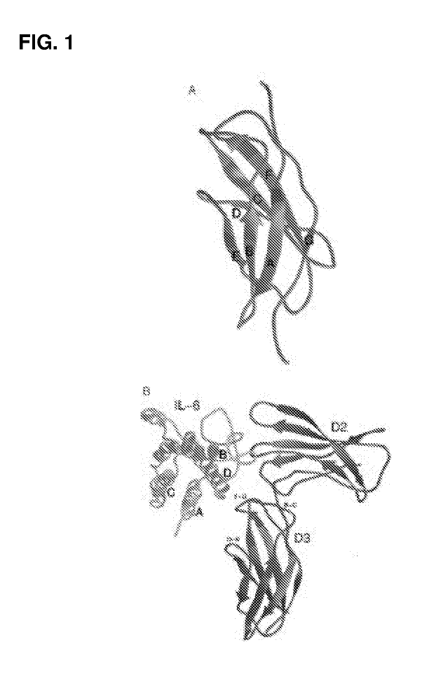

We hypothesize that the above-mentioned genetic markers are, to various extents, linked to a genuine determinant of the binding of TCZ to the IL-6R. According to Makoto et al., TCZ (previously known as MRA) is likely to bind within or near the pocket binding IL-6, consisting of B′-C′ loop and F′-G′ loop shown in FIG. 1.

Given that different allotypes of the IL-6R exist, and given that the mouse from which the MRA clone originated had been immunized with only one allotype of the interleukin-6-receptor (IL-6R), TCZ would have high affinity for the allotype in question, but not necessarily for other allotypes. Even if the mouse had been immunized with a mixture of IL-6R allotypes, it might well produce different B cells that react with different IL-6R allotypes, but the monoclonal nature of TCZ would still imply that a clone specific for one particular allotype has been selected.

Therefore, SNPs within, or in proximity to, the DNA sequences encoding the B′-C′ loop or the F′-G′ loop may change the affinity of the antibody to the IL-6R. In other words, TCZ may have a strong preference for binding to certain allotypes of its target molecule, in analogy to what has been suggested for other therapeutic antibodies.

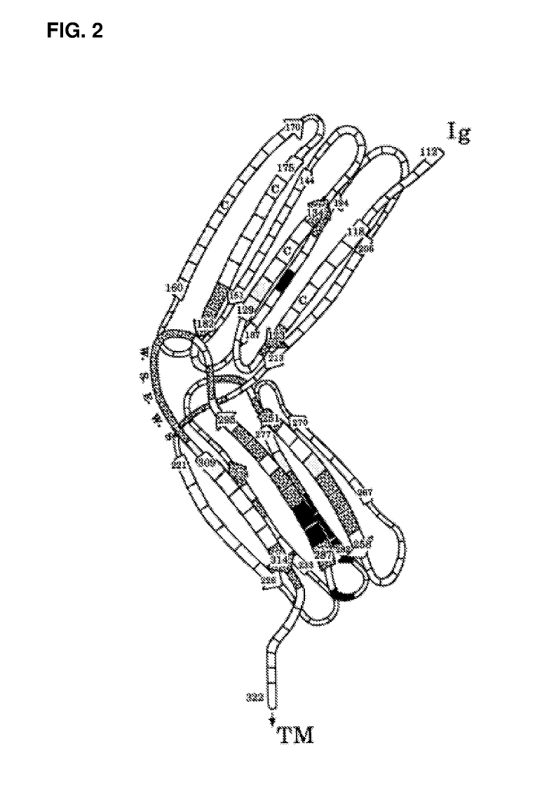

According to Yawata et al., the B′-C′ loop and the F′-G′ loop involve amino acid residues 252-55 and 297-307 (see FIGS. 1 and 2). Three polymorphisms that have not been tested so far encode amino acids in the extracellular region of the IL-6R are candidates for SNPs with high predictive value. These are rs8192282 resulting in a synonymous substitution (Ala31Ala), rs11557725 resulting in a Leu81Arg substitution, and rs11557724 resulting in a Pro136Ser substitution.

### Hypothesis 1

We hypothesize that the rs12083537, rs7526293, rs4509570 and the rs2228145/rs4129267 polymorphisms, all of which are associated with the best clinical responses in the group of heterozygotes, are better predictors of the response to TCZ when used in combination with one or more other IL6R genetic variations, such as one or more other IL6R SNPs than alone.

Hasan et al. have previously investigated whether the sIL-6R Asp358Ala polymorphism (corresponding to rs2228145) influenced the response to anti-TNF therapy in patients with rheumatoid arthritis, and showed that it did not. Marinou et al. investigated whether the same polymorphism influenced RA susceptibility and failed to show that it did (p=0.08). They confirmed a gene-dose association between IL-6R A358D genotypes and sIL-6R levels in a healthy British population and found that low sIL-6R levels are a risk factor for the development of RA. Marinou et al recognized, however, that 49% of the variance in sIL-6R levels in European Americans is attributable to the rs8192284 (corresponding to rs2228145) genotype. The authors of both papers put their findings into perspective by suggesting that the Asp358Ala polymorphism may be useful as a marker predicting the response to TCZ. However, neither of the papers present any data supporting this notion. Neither do any of them specify whether the A/A-, C/A- or C/C-genotypes potentially confer a risk for lack of response to TCZ.

### Hypothesis 2

We hypothesize that the SNPs rs8192282 (resulting in the synonymous substitution Ala31Ala), rs11557725 (resulting in a Leu81Arg substitution), and rs11557724 (resulting in a Pro136Ser substitution) are excellent candidates for predicting the response to TCZ.

### Hypothesis 3

We hypothesize that genetic variations in the IL-6 gene other than SNPs may determine the efficiency of TCZ and thus be helpful in predicting the response of patients to IL6R inhibitors such as the IL6R antibody TCZ, cf. Example 3.

### Example 3

### Full Length Sequencing of IL6R

Beside SNPs, several different classes of genetic variation exists, i.e. copy-number variations (CNV), insertions/deletions (indels), and microsatellites. These forms of genetic variations may also be accountable for the outcome of therapy with IL6R inhibitors such as TCZ, either alone or in combination.

The SNPs listed herein account only for already known genetic variation, and as such does not necessarilly cover rare or population-specific SNPs nor CNVs, indels, etc. However, CNVs, indels, and rare SNPs may have an influence on the outcome of IL6R inhibitor therapy, e.g. one or more of the above mentioned types of genetic variation in IL6R either alone or in combination may influence the outcome of treatment with IL6R inhibitors such as TCZ by e.g. increasing or reducing the concentration of soluble and/or membrane-bound IL-6-receptor, or by changing the conformation and/or structure of the receptor thereby affecting the ability of the inhibitor to neutralize it. For rare SNPs, it is furthermore conceivable that a person can have one or even a few rare SNPs in the IL6R region inhibiting (or improving) their response to a particular IL6R inhibitor such as TCZ. In this way, several persons may exhibit a poorer (or better) response to a particular IL6R inhibitor without necessarily carrying identical polymorphisms. Many rare or de novo polymorphisms are expected to exist but have not necessarily been reported to genetic variation databases.

Sequencing the entire IL6R gene-region in TCZ treated patients may thus provide us with additional information about CNVs, insertions/deletions, rare SNPs, novel and/or population-specific variations, etc., as well as genotypes for all common SNPs in the area, which may be directly applied as prognostic markers of the outcome of TCZ therapy by comparing these data for patients with poor versus patients with favourable outcome of TCZ therapy.

The information obtained from sequencing the IL6R gene-region may likewise be useful for predicting the response of patients to IL6R inhibitors different from TCZ.

Sequencing can be performed in several different ways, the gold-standard currently being the Sanger method using chain-termination chemistry. However, the so-called next generation sequencing methods are evolving quickly and are capable of sequencing very large areas of the genome rapidly. For deletions, insertions, and SNPs, both Sanger sequencing and next-generation sequencing methods can be applied, but while Sanger sequencing is a laborious and time consuming method, next-generation sequencing techniques offer the ability to sequence entire genes in one run making these the preferred choice when trying to discover new polymorphisms. Copy-number variations (CNVs) can comprise copies or deletions of very large segments of the genome for which quantitative methods such as TaqMan, molecular beacons, etc. are well-suited. Depending on their exact sizes, both Sanger sequencing and next-generation sequencing methods can be well-suited for detecting smaller CNVs, microsattelites etc. For already known SNPs, insertions, and deletions, both TaqMan, molecular beacons, RFLP, qPCR, and various bead-based techniques, as already described herein are well-suited.

Genomic DNA may be obtained from TCZ treated patients from anti-coagulated blood samples by means of a commercially available DNA-purification kit, such as the Maxwell® 16 Blood DNA Purification Kit (Promega Corporation) intended for use on the Maxwell® 16 Instrument (Promega Corporation).

Sequencing of the obtained genomic DNA can subsequently be performed by the Sanger method. In this method, the DNA sequence of the IL6R including e.g. 5 kilobases up and downstream of the gene, is amplified by means of a polymerase chain-reaction using commercially available kits such as Qiagen Multiplex Master Mix (Qiagen) on a thermocycler such as a GeneAmp 9600 (Perkin Elmer) employing a program consisting of an initial 95 C. hot-start for 15 min. followed by 35 cycles consisting of a 94 C. denaturing step for 90 sec., a 60 C primer annealing step for 90 sec, and a 72 C. elongation step for 90 sec. Subsequently, the cycling sequence is followed by a final 68 C. elongation step for 15 min. and the reactions kept at −18 C. until further use.

Each PCR-reaction consists of 12.5 uL 2× Qiagen Multiplex PCR Mastermix, 2.5 uL genomic template DNA, 5.0 uL of a 1 pmol/uL of each PCR-primer sequence, specific for a particular region of the IL6R gene sequence, and 5 uL of water.

After thermocycling, the PCR-products are purified using commercially available kits such as the Wizard® SV Gel and PCR Clean-Up System (Promega Corporation).

Purified PCR-products are quantified and sequenced on a sequencing machine such as the ABI PRISM 3700 DNA Analyzer (Applied Biosystems) based on the principles of the Sanger sequencing method.

Obtained IL6R-DNA sequences are subsequently compared to the IL6R reference sequence (NG_{—}012087.1) for detection of genetic variations, including but not limited to: Copy-number variations (CNV), insertions, deletions, single nucleotide polymorphisms, repeats, etc.

Presence or absence of the detected genetic variations in the obtained patient IL6R-DNA sequences are then compared between TCZ treatment responders and non-responders to assess their influence on TCZ treatment-outcome.

The preferred clinical parameter for assessing TCZ therapy-outcome is the change in the patients number of swollen joints since this appears to be the most objective parameter available.

1. A method for predicting the response of a patient to treatment with an Interleukin 6 Receptor inhibitor comprising the analysis of one or more genetic variations in the Interleukin 6 Receptor gene, the method comprising the steps of:
i) obtaining a sample of genomic DNA from the patient;
ii) detecting at least one genetic variation in the Interleukin 6 Receptor gene in said sample; and
iii) predicting whether said patient is a responder, a non-responder or a partial responder.

2. The method of claim 1, wherein the Interleukin 6 Receptor inhibitor is an antibody directed against the Interleukin 6 Receptor.

3. The method of claim 2, wherein the antibody is Tocilizumab.

4. The method of claim 1, wherein the one or more genetic variations are selected from the group consisting of one or more single nucleotide polymorphisms, one or more copy number variations, one or more insertions/deletions, and one or more microsatellites.

5. The method of claim 0, wherein said genetic variation is one or more single nucleotide polymorphisms, said polymorphism selected from the group consisting of rs12083537, rs7526293, rs2228145, rs4129267, rs4509570, rs8192282, rs11557725, rs11557724, rs952146, rs17654071, rs6427631, rs2054855, rs4845615, rs1552481, rs4845617, rs746475, rs6427641, rs11265610, rs1386821, rs7411976, rs4075016, rs4075015, rs12090237, rs4601580, rs6684439, rs4845618, rs6427658, rs6694817, rs28730733, rs7549250, rs7553796, rs7518199, rs4845371, rs6667434, rs4553185, rs4845622, rs4393147, rs4453032, rs6664201, rs4845623, rs4537545, rs7529229, rs4845625, rs4845626, rs6689393, rs4845374, rs28730735, rs28730736, rs11265618, rs12125954, rs10159236, rs10752641, rs4329505, rs4240872, rs4341355, rs2229238, rs7514452, rs4072391, rs4379670 and rs11265621.

6. The method of claim 1, wherein the one or more genetic variations is localised in a region of the Interleukin 6 Receptor gene selected from the group consisting of: coding region of the Interleukin 6 Receptor gene, non-coding region of the Interleukin 6 Receptor gene, promoter region, exons, introns, inside the binding region of the inhibitor, outside the binding region of the inhibitor, upstream untranslated region and downstream untranslated region.

7-8. (canceled)

9. The method of claim 5, wherein the single nucleotide polymorphism rs12083537 either alone or in combination with one or more other genetic variations in the Interleukin 6 Receptor gene is used to predict the subset of patients which are responders to Tocilizumab, said responders being heterozygous for rs12083537.

10. The method of claim 5, wherein the single nucleotide polymorphism rs7526293 either alone or in combination with one or more other genetic variations in the Interleukin 6 Receptor gene is used to predict the subset of patients which are responders to Tocilizumab, said responders being heterozygous for rs7526293.

11. The method of claim 5, wherein the single nucleotide polymorphism rs4509570 either alone or in combination with one or more other genetic variations in the Interleukin 6 Receptor gene is used to predict the subset of patients which are responders to Tocilizumab, said responders being heterozygous for rs4509570.

12. The method of claim 5, wherein the single nucleotide polymorphism rs2228145 or rs4129267 either alone or in combination with one or more other genetic variations in the Interleukin 6 Receptor gene is used to predict the subset of patients which are responders to Tocilizumab, said responders being heterozygous for rs2228145 or rs4129267.

13. The method of claim 5, wherein the single nucleotide polymorphism is rs8192282.

14. The method of claim 5, wherein the single nucleotide polymorphism is rs11557725.

15. The method of claim 5, wherein the single nucleotide polymorphism is rs11577724.

16. The method of claim 1, wherein two to four genetic variations in the Interleukin 6 Receptor are analyzed.

17. The method of claim 1, wherein the patient is suffering from a disease involving the Interleukin 6 Receptor, said disease being treatable with an inhibitor of the Interleukin 6 Receptor.

18. The method of claim 0, wherein the disease is rheumatoid arthritis.

19. (canceled)

20. The method according to claim 1, wherein the method is an in vitro diagnostic method.

21. A kit for use in a method for predicting the response of a patient to an inhibitor of the Interleukin 6 Receptor according to claim 1, said kit comprising a means for detecting at least one genetic variation in the Interleukin 6 Receptor gene, such as a single nucleotide polymorphism.

22. The kit according to claim 0, wherein the inhibitor of the Interleukin 6 Receptor is Tocilizumab.

23. A method for treatment of a patient for a disease involving the IL6R, said patient predicted to be responder or a partial responder to Tocilizumab, comprising: administering Tocilizumab to said patient.

24. The method according to claim 23, wherein the disease involving the IL6R is selected from the group consisting of rheumatoid arthritis, multiple myeloma, juvenile chronic arthritis, osteoarthritis, asthma, chrohn's disease, interstitial lung disease, inflammatory bowel disease, systemic sclerosis, intraocular inflammation, graves' disease and endometriosis.


SEQUENCE LISTING (76 sequences)

SEQ ID NO: 1 - DNA, 20 bp, Artificial sequence
  PCR-primer
gcttttgacagcaccagcta

SEQ ID NO: 2 - DNA, 20 bp, Artificial sequence
  PCR-primer
agctgctggcagtgttacag

SEQ ID NO: 3 - DNA, 19 bp, Artificial sequence
  PCR-primer
gggtagagccggaagacaa

SEQ ID NO: 4 - DNA, 20 bp, Artificial sequence
  PCR-primer
cacctgcaatgctttccttt

SEQ ID NO: 5 - DNA, 20 bp, Artificial sequence
  PCR-sequence
gtaccactgcccacattcct

SEQ ID NO: 6 - DNA, 20 bp, Artificial sequence
  PCR-primer
gtaccactgcccacattcct

SEQ ID NO: 7 - DNA, 20 bp, Artificial sequence
  PCR-primer
ggacgagtgatgggtagcat

SEQ ID NO: 8 - DNA, 20 bp, Artificial sequence
  PCR-primer
attccccaagaatgggaatc

SEQ ID NO: 9 - DNA, 18 bp, Artificial sequence
  PCR-primer
tgggcagcagaaggagac

SEQ ID NO: 10 - DNA, 21 bp, Artificial sequence
  PCR-primer
cccagtttttgatgacaatcc

SEQ ID NO: 11 - DNA, 20 bp, Artificial sequence
  PCR-primer
ccagagtggagcactgtgaa

SEQ ID NO: 12 - DNA, 21 bp, Artificial sequence
  PCR-primer
ccaccttccagaagcacataa

SEQ ID NO: 13 - DNA, 20 bp, Artificial sequence
  PCR-primer
ttctcgaggttgaagcaaca

SEQ ID NO: 14 - DNA, 20 bp, Artificial sequence
  PCR-primer
gggtagggggtggttagaga

SEQ ID NO: 15 - DNA, 20 bp, Artificial sequence
  PCR-primer
cagtgtgaggcctatgctga

SEQ ID NO: 16 - DNA, 20 bp, Artificial sequence
  PCR-primer
ccctctccctccttcctaga

SEQ ID NO: 17 - DNA, 20 bp, Artificial sequence
  PCR-primer
ccacctcccaagttcaagtg

SEQ ID NO: 18 - DNA, 20 bp, Artificial sequence
  PCR-primer
acaatggcaggcagaggtag

SEQ ID NO: 19 - DNA, 20 bp, Artificial sequence
  PCR-primer
cacaaacgatcctcccatct

SEQ ID NO: 20 - DNA, 20 bp, Artificial sequence
  PCR-primer
ggacccatctcacctcagaa

SEQ ID NO: 21 - DNA, 20 bp, Artificial sequence
  PCR-primer
ctccacacacgttccctctt

SEQ ID NO: 22 - DNA, 20 bp, Artificial sequence
  PCR-primer
cacaactcaccatccaccag

SEQ ID NO: 23 - DNA, 20 bp, Artificial sequence
  PCR-primer
agccaacatgcacatagctc

SEQ ID NO: 24 - DNA, 20 bp, Artificial sequence
  PCR-primer
aaaacacggcttggcataat

SEQ ID NO: 25 - DNA, 20 bp, Artificial sequence
  PCR-primer
aaaacacggcttggcataat

SEQ ID NO: 26 - DNA, 20 bp, Artificial sequence
  PCR-primer
tccaagatttcggctacctg

SEQ ID NO: 27 - DNA, 20 bp, Artificial sequence
  PCR-primer
ccggaaatgtgtaaccaagg

SEQ ID NO: 28 - DNA, 20 bp, Artificial sequence
  PCR-primer
gggactctcagggtgtgtgt

SEQ ID NO: 29 - DNA, 20 bp, Artificial sequence
  PCR-primer
tcccatgatgcagtgagcta

SEQ ID NO: 30 - DNA, 20 bp, Artificial sequence
  PCR-primer
tcaactgccttcctgttcct

SEQ ID NO: 31 - DNA, 20 bp, Artificial sequence
  PCR-primer
ctgctcattttggtggatga

SEQ ID NO: 32 - DNA, 20 bp, Artificial sequence
  PCR-primer
gcaactttccctgtgctacc

SEQ ID NO: 33 - DNA, 20 bp, Artificial sequence
  PCR-primer
gggaccaggattcaggagac

SEQ ID NO: 34 - DNA, 20 bp, Artificial sequence
  PCR-primer
ttccttgatggtgatgtgga

SEQ ID NO: 35 - DNA, 22 bp, Artificial sequence
  PCR-primer
aagtgtcctctccctacctctg

SEQ ID NO: 36 - DNA, 21 bp, Artificial sequence
  PCR-primer
ttttcagcaccataggaaagg

SEQ ID NO: 37 - DNA, 22 bp, Artificial sequence
  PCR-primer
ttgtgaccagaaatgtcgtaga

SEQ ID NO: 38 - DNA, 20 bp, Artificial sequence
  PCR-primer
aactgagattgcaccattgc

SEQ ID NO: 39 - DNA, 47 bp, Artificial sequence
  ASPE-primer
ctttaatcctttatcactttatcagggcagtggtactgaagaagaat

SEQ ID NO: 40 - DNA, 47 bp, Artificial sequence
  ASPE-primer
cttttcaattacttcaaatcttcagggcagtggtactgaagaagaag

SEQ ID NO: 41 - DNA, 45 bp, Artificial sequence
  ASPE-primer
ctttatcaatacatactacaatcacttggagtggggtcaattctc

SEQ ID NO: 42 - DNA, 45 bp, Artificial sequence
  ASPE-primer
atactaactcaactaactttaaaccttggagtggggtcaattctt

SEQ ID NO: 43 - DNA, 41 bp, Artificial sequence
  ASPE-primer
ttacctttatacctttctttttaccaccgacctcagcagca

SEQ ID NO: 44 - DNA, 41 bp, Artificial sequence
  ASPE-primer
cttctcattaacttacttcataatcaccgacctcagcagcc

SEQ ID NO: 45 - DNA, 40 bp, Artificial sequence
  ASPE-primer
caatttcatcattcattcatttcaatggggtgctccgagg

SEQ ID NO: 46 - DNA, 41 bp, Artificial sequence
  ASPE-primer
taatcttctatatcaacatcttacgatggggtgctccgaga

SEQ ID NO: 47 - DNA, 43 bp, Artificial sequence
  ASPE-primer
aatcatacctttcaatcttttacacaatggcaatgcagaggag

SEQ ID NO: 48 - DNA, 43 bp, Artificial sequence
  ASPE-primer
ctttctatctttctactcaataatcaatggcaatgcagaggaa

SEQ ID NO: 49 - DNA, 45 bp, Artificial sequence
  ASPE-primer
atacttcattcattcatcaattcaggacccatctcacctcagaac

SEQ ID NO: 50 - DNA, 45 bp, Artificial sequence
  ASPE-primer
aatctacactaacaatttcataacggacccatctcacctcagaat

SEQ ID NO: 51 - DNA, 50 bp, Artificial sequence
  ASPE-primer
ctttcaattacaatactcattacacaaaaatattaaatgggaaattccag

SEQ ID NO: 52 - DNA, 50 bp, Artificial sequence
  ASPE-primer
tacaaatcatcaatcactttaatccaaaaatattaaatgggaaattccat

SEQ ID NO: 53 - DNA, 47 bp, Artificial sequence
  ASPE-primer
atcaaatctcatcaattcaacaataaacaaactcctccagaatggat

SEQ ID NO: 54 - DNA, 47 bp, Artificial sequence
  ASPE-primer
caattcatttaccaatttaccaataaacaaactcctccagaatggac

SEQ ID NO: 55 - DNA, 47 bp, Artificial sequence
  ASPE-primer
ctatctttaaactacaaatctaacggagtttttggtttgattcttgg

SEQ ID NO: 56 - DNA, 48 bp, Artificial sequence
  ASPE-primer
caatatcatcatctttatcattacaggagtttttggtttgattcttgt

SEQ ID NO: 57 - DNA, 52 bp, Artificial sequence
  ASPE-primer
ctacaaacaaacaaacattatcaaacccttaatttaataatgcatacacctg

SEQ ID NO: 58 - DNA, 53 bp, Artificial sequence
  ASPE-primer
aatcatactcaactaatcattcaacacccttaatttaataatgcatacaccta

SEQ ID NO: 59 - DNA, 47 bp, Artificial sequence
  ASPE-primer
ataccaataatccaattcatatcagagttcccacagactcaactgtg

SEQ ID NO: 60 - DNA, 47 bp, Artificial sequence
  ASPE-primer
aaacaaacttcacatctcaataatgagttcccacagactcaactgtc

SEQ ID NO: 61 - DNA, 43 bp, Artificial sequence
  ASPE-primer
tcaaaatctcaaatactcaaatcaagtgcatggtgtcaggacc

SEQ ID NO: 62 - DNA, 44 bp, Artificial sequence
  ASPE-primer
tcaatcataatctcataatccaatcagtgcatggtgtcaggact

SEQ ID NO: 63 - DNA, 43 bp, Artificial sequence
  ASPE-primer
aatcctttctttaatctcaaatcaacatgagcaaatggtggga

SEQ ID NO: 64 - DNA, 43 bp, Artificial sequence
  ASPE-primer
ctactaattcattaacattactacacatgagcaaatggtgggg

SEQ ID NO: 65 - DNA, 48 bp, Artificial sequence
  ASPE-primer
aatctaacaaactcatctaaatacgtgaggggagtatttgtttaccct

SEQ ID NO: 66 - DNA, 46 bp, Artificial sequence
  ASPE-primer
tcaactaactaatcatctatcaatgaggggagtatttgtttacccc

SEQ ID NO: 67 - DNA, 40 bp, Artificial sequence
  ASPE-primer
tcattcatatacataccaattcatgggtggggatggctct

SEQ ID NO: 68 - DNA, 40 bp, Artificial sequence
  ASPE-primer
tcaatcatctttatacttcacaatgggtggggatggctca

SEQ ID NO: 69 - DNA, 47 bp, Artificial sequence
  ASPE-primer
caataaactatacttcttcactaatccaaatgatggtgttagaggtg

SEQ ID NO: 70 - DNA, 47 bp, Artificial sequence
  ASPE-primer
ctatctatctaactatctatatcatccaaatgatggtgttagaggta

SEQ ID NO: 71 - DNA, 55 bp, Artificial sequence
  ASPE-primer
ctactatacatcttactatacttttaatttttgtaattttagtagcaatgaggtt

SEQ ID NO: 72 - DNA, 55 bp, Artificial sequence
  ASPE-primer
aatcaatcttcattcaaatcatcataatttttgtaattttagtagcaatgaggtc

SEQ ID NO: 73 - DNA, 52 bp, Artificial sequence
  ASPE-primer
aatctcataatctacatacactatgtactagctgacttaacagtttcagtcg

SEQ ID NO: 74 - DNA, 53 bp, Artificial sequence
  ASPE-primer
ttcaatcattcaaatctcaacttttgtactagctgacttaacagtttcagtca

SEQ ID NO: 75 - DNA, 50 bp, Artificial sequence
  ASPE-primer
tcaattaccttttcaatacaatactggtgactctcaattctttttttttt

SEQ ID NO: 76 - DNA, 50 bp, Artificial sequence
  ASPE-primer
aatcctttttactcaattcaatcatggtgactctcaattcttttttttta